(19) United States
(12) Patent Application Publication
(10) Pub. No.: US 2026/0140358 A1
(43) Pub. Date: May 21, 2026

(54) MOTION PLATFORM

(71) Applicant: Bionano Genomics, Inc., San Diego, CA (US)

(72) Inventors: Werner WILLEMSE, San Diego, CA (US); Paul RODER, San Diego, CA (US)

(21) Appl. No.: 19/119,757

(22) PCT Filed: Oct. 10, 2023
(86) PCT No.: PCT/US2023/076465

§ 371 Date: Apr. 9, 2025

Related U.S. Application Data
(60) Provisional application No. 63/414,852, filed on Oct. 10, 2022.
(60) Provisional application No. 63/414,858, filed on Oct. 10, 2022.
(60) Provisional application No. 63/515,334, filed on Jul. 24, 2023.
(60) Provisional application No. 63/515,343, filed on Jul. 25, 2023.

(51) Int. Cl. G02B 21/26; G01B 11/27; G01N 21/64; G01N 33/53; G02B 21/32

(52) U.S. Cl. CPC G02B 21/26; G01B 11/27; G01N 21/6428; G01N 33/5308; G02B 21/32; G01N 2021/6439

(57) ABSTRACT

Disclosed herein include a motion platform comprising a base; an x-y motion stage on the base; a tip-axis adjustment engagement component (e.g., a pawl) and/or a tilt-axis adjustment pawl engagement component (e.g., a pawl) on the base; a tip and tilt (TnT) motion stage on the x-y motion stage. The TnT motion stage can comprise: a sample carrier, two tip-axis goniometers with different slopes relative to one axis of the x-axis and the y-axis, and/or a tilt-axis slanted linear bearing. The TnT motion stage can comprise: a tip-axis complementary adjustment engagement component (e.g., a notch), and a tilt-axis complementary adjustment engagement component (e.g., a notch). In some embodiments, when the tip-axis adjustment engagement component is engaged with the tip-axis complementary adjustment engagement component, a movement of the x-y motion stage along the one axis results in a movement of the TnT motion stage along the two tip-axis goniometers, thereby resulting in a change in the tip of the TnT motion stage. In some embodiments, when the tilt-axis adjustment engagement component is engaged with the tilt-axis complementary adjustment engagement component, a movement of the x-y motion stage along the one axis results in a movement of the TnT motion stage along the tilt-axis slanted linear bearing, thereby resulting in a change in the tilt of the TnT motion stage.

### RELATED APPLICATIONS

The present application is a U.S. national phase application under 35 U.S.C. § 371 of International Application No. PCT/US2023/076465, filed on Oct. 10, 2023 and published as WO 2024/081644 A1 on Apr. 18, 2024, which claims priority under 35 U.S.C. § 119 (e) to U.S. Provisional Application No. 63/414,852, filed Oct. 10, 2022, U.S. Provisional Application No. 63/414,858, filed Oct. 10, 2022, U.S. Provisional Application No. 63/515,334, filed Jul. 24, 2023, and U.S. Provisional Application No. 63/515,343, filed Jul. 25, 2023. The content of each of the related applications is incorporated herein by reference in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field

This disclosure relates generally to the field of motion platforms, and more particularly to motion platforms for microscopy.

### Background

Microscopy instruments, such as fluorescent imaging instruments, (e.g., optical genome mapping (OGM)) instruments), require high-magnification optics to produce images with sufficiently resolution (e.g., images with sufficiently resolution of the molecules being imaged, such as DNA molecules being imaged). High-magnification optics can have a narrow depth-of-focus. Microscopy instruments can raster scan many successive images. Throughput of instruments are generally proportionate to the Field of View (FoV) size of images acquired. There is a need to decrease (e.g., minimize) move times between FoVs to increase (e.g., maximize) instrument throughput.

### SUMMARY

Disclosed herein includes embodiments of a motion platform. In some embodiments, a motion platform can comprise: a base. The motion platform can comprise: an x-y motion stage. The x-y motion stage can be on (e.g., attached to, such as securely attached to) the base. The motion platform can comprise: a tip-axis adjustment pawl (or a tip-axis adjustment engagement component) on the base. The motion platform can comprise: a tilt-axis adjustment pawl (or a tilt-axis adjustment engagement component) on (e.g., attached to, such as securely attached to) the base. The motion platform can comprise: a tip and tilt (TnT) motion stage. The TnT motion stage can be on the x-y motion stage. The TnT motion stage can comprise: two tip-axis goniometers with different slopes (e.g., 3.6° and −3.6° respectively as illustrated in FIG. 2) relative to one axis (e.g., x-axis) of the x-axis and the y-axis (e.g., of the motion platform or the motion stage). The TnT motion stage can comprise: a bearing, such as a tilt-axis slanted linear bearing (e.g., slanted relative to the plane of the platform and/or the x-y motion stage). The bearing can be a recirculating linear bearing. The bearing can be a non-recirculating linear bearing. The TnT motion stage can comprise: a tip-axis adjustment notch (or a tip-axis complementary adjustment engagement component). The TnT motion stage can comprise: a tilt-axis adjustment notch (or a tilt-axis complementary adjustment engagement component). In some embodiments, the TnT motion stage can comprise: a sample carrier. In some embodiments, when the tip-axis adjustment pawl is engaged with the tip-axis adjustment notch, a movement of the x-y motion stage along the one axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

Disclosed herein include embodiments of a motion platform. In some embodiments, a motion platform can comprise: a base. The motion platform can comprise: an x-y motion stage on the base. The motion platform can comprise: a tip-axis adjustment pawl (or a tip-axis adjustment engagement component) on the base. The motion platform can comprise: a tip motion stage. The tip motion stage can be on the x-y motion stage. The tip motion stage can comprise: one or more (e.g., 2) tip-axis goniometers with different slopes relative to one axis of the x-axis and the y-axis. The tip motion stage can comprise: a tip-axis adjustment notch. In some embodiments, when the tip-axis adjustment pawl is engaged with the tip-axis adjustment notch, a movement of the x-y motion stage along the one axis results in a movement of the tip motion stage along the one or more tip-axis goniometers. This can result in a change in the tip of the tip motion stage. In some embodiments, the tip motion stage can comprise: a sample carrier.

In some embodiments, the motion platform further comprises: a tilt-axis adjustment pawl (or a tilt-axis adjustment engagement component) on the base. The tip motion stage can be a tip and tilt (TnT) motion stage. The TnT motion stage can further comprise: a tilt-axis slanted linear bearing. The TnT motion stage can further comprise: a tilt-axis adjustment notch (or a tilt-axis complementary adjustment engagement component). In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

Disclosed herein include embodiments of a motion platform. In some embodiments, a motion platform can comprise: a base. The motion platform can comprise: an x-y motion stage on the base. The motion platform can comprise: a tilt-axis adjustment pawl (or a tilt-axis adjustment engagement component) on the base. The motion platform can comprise: a tilt motion stage. The tilt motion stage can be on the x-y motion stage. The tilt motion stage can comprise: a tilt-axis slanted linear bearing. The tilt motion stage can comprise: a tilt-axis adjustment notch (or a tilt-axis complementary adjustment engagement component). In some embodiments, when the tilt-axis adjustment pawl is engaged with the tilt-axis adjustment notch, a movement of the x-y motion stage along the one axis results in a movement of the tilt motion stage along the tilt-axis slanted linear bearing. This can result in a change in the tilt of the tilt motion stage. In some embodiments, the tilt motion stage can comprise: a sample carrier.

In some embodiments, the motion platform further comprises: a tip-axis adjustment pawl on the base. The tilt motion stage can be a tip and tilt (TnT) motion stage. The TnT motion stage can further comprise: one or more (e.g., 2) tip-axis goniometers with different slopes relative to one axis of the x-axis and the y-axis. The TnT motion stage can further comprise: a tip-axis adjustment notch (or a tip-axis complementary adjustment engagement component). In some embodiments, when the tip-axis adjustment pawl is engaged with the tip-axis adjustment notch, a movement of the x-y motion stage along the one axis results in a movement of the TnT motion stage along the one or more tip-axis goniometers. This can result in a change in the tip of the TnT motion stage.

Disclosed herein include embodiments of a motion platform. In some embodiments, a motion platform can comprise: a base. The motion platform can comprise: an x-y motion stage. The x-y motion stage can be on (e.g., attached to, such as securely attached to) the base. The motion platform can comprise: a first-axis (e.g., a tip-axis or a tilt-axis) adjustment engagement component (e.g., a pawl or a notch) on (e.g., attached to, such as securely attached to) the base. The motion platform can comprise: a motion stage (e.g., a tip motion stage, a tilt motion stage, or a tip and tilt (TnT) motion stage). The second motion stage can be on the x-y motion stage. The motion platform can comprise: a first-axis goniometer or bearing. The motion platform can comprise: a first-axis complementary adjustment engagement component (e.g., a notch or a pawl), The first-axis adjustment engagement component and the first-axis complementary adjustment engagement component can engage (or be in engagement, such as secure engagement) with each other. For example, the first-axis adjustment engagement component and the first-axis complementary engagement component can be a pawl and a notch and can be in engagement (e.g., secure engagement) with each other. When the first-axis adjustment component is engaged with the first-axis complementary adjustment engagement component, a movement of the x-y motion stage along one axis (e.g., x-axis) of the x-axis and the y-axis can result. in a movement of the second motion stage along the first-axis goniometer or bearing. This can result in a change in the first-axis (e.g., tip-axis) of the second motion stage. In some embodiments, the motion platform can comprise: a sample carrier.

In some embodiments, the motion platform can further comprise a second-axis (e.g., a tilt-axis or a tip-axis) adjustment engagement component (e.g., a pawl or a notch). The second motion stage can further comprise: a second-axis goniometer or bearing. The second motion stage can further comprise: a second-axis complementary adjustment engagement component (e.g., a notch or a pawl). The second-axis adjustment engagement component and the second-axis complementary adjustment engagement component can engage (or be in engagement, such as secure engagement) with each other. For example, the second-axis adjustment engagement component and the second-axis complementary engagement component can be a pawl and a notch and can be in engagement (e.g., secure engagement) with each other. When the second-axis adjustment engagement component is engaged with the second-axis complementary adjustment engagement component, a movement of the x-y motion stage along the one axis can result in a movement of the second motion stage along the second-axis goniometer or bearing. This can result in a change in the second-axis (e.g., tilt-axis) of the second motion stage.

In some embodiments, the first-axis is the tip-axis. The second-axis can be the tilt-axis. In some embodiments, the first-axis is the tilt-axis. The second-axis can be the tip-axis. In some embodiments, the first-axis goniometer can comprise one or more (e.g., 2) first-axis goniometers. In some embodiments, the first-axis bearing comprises a first-axis slanted linear bearing.

In some embodiments, the motion platform comprises an x-axis motor on (e.g., attached to, such as securely attached to) the base. The motion platform can comprise a y-axis motor on (e.g., attached to, such as securely attached to) the base. The x-axis motor can move the x-y motion stage along the x-axis. The y-axis motor can move the x-y motion stage along the y-axis. The motion platform can comprise no additional motor other than the x-axis motor and the y-axis motor for changing the tip and/or tilt of the TnT motion stage. In some embodiments, the x-axis motor is a servomotor. The y-axis motor can be a servomotor.

In some embodiments, the tip-axis adjustment pawl and the tilt-axis adjustment pawl point in the opposite directions. In some embodiments, the tip-axis adjustment notch and the tilt-axis adjustment notch point in the opposite directions. In some embodiments, the tip-axis adjustment pawl and the tilt-axis adjustment pawl are elevated from the base. In some embodiments, tip-axis adjustment pawl and the tilt-axis adjustment pawl are at different heights relative to the base. The tilt-axis adjustment notch and the tip-axis adjustment notch can be at different heights relative to the base. In some embodiments, the tip-axis adjustment pawl and the tilt-axis adjustment pawl are at an identical height relative to the base. The tilt-axis adjustment notch and the tip-axis adjustment notch can be at an identical height relative to the base.

In some embodiments, the TnT motion stage comprises no motor. In some embodiments, the TnT motion stage is in contact with the x-y motion stage via the two tip-axis goniometers and the tilt-axis slanted linear bearing. In some embodiments, the TnT motion stage is in contact with the x-y motion stage only via the two tip-axis goniometers and the tilt-axis slanted linear bearing.

In some embodiments, the TnT motion stage comprises no motor. In some embodiments, the TnT motion stage is in contact with the x-y motion stage via the one or more first-axis goniometers and the second-axis bearing. In some embodiments, the TnT motion stage is in contact with the x-y motion stage only via the one or more first-axis goniometers and the second-axis bearing.

In some embodiments, the two tip-axis goniometers have different slopes relative to the x-axis (or the y-axis). In some embodiments, the angle of the slope of the other of the two tip-axis goniometers can be, be about, be at least, be at least about, be at most, or be at most about, ±1°, ±1.1°, ±1.2°, ±1.3°, ±1.4°, ±1.5°, ±1.6°, ±1.7°, ±1.8°, ±1.9°, ±2°, ±2.1°, ±2.2°, ±2.3°, ±2.4°, ±2.5°, ±2.6°, ±2.7°, ±2.8°, ±2.9°, ±3.0°, ±3.1°, ±3.2°, ±3.3°, ±3.4°, ±3.5°, ±3.6°, ±3.7°, ±3.8°, ±3.9°, ±4°, ±4.1°, ±4.2°, ±4.3°, ±4.4°, ±4.5°, ±4.6°, ±4.7°, ±4.8°, ±4.9°, ±5°, ±5.1°, ±5.2°, ±5.3°, ±5.4°, ±5.5°, ±5.6°, ±5.7°, ±5.8°, ±5.9°, ±6°, ±6.1°, ±6.2°, ±6.3°, ±6.4°, ±6.5°, ±6.6°, ±6.7°, ±6.8°, ±6.9°, ±7°, ±7.1°, ±7.2°, ±7.3°, ±7.4°, ±7.5°, ±7.6°, ±7.7°, ±7.8°, ±7.9°, ±8°, ±8.1°, ±8.2°, ±8.3°, ±8.4°, ±8.5°, ±8.6°, ±8.7°, ±8.8°, ±8.9°, ±9°, ±9.1.°, ±9.2°, ±9.3°, ±9.4°, ±9.5°, ±9.6°, ±9.7°, ±9.8°, ±9.9°, ±10°, or a number or a range between any two of these values. In some embodiments, the slopes of the two tip-axis goniometers have different absolute angles. In some embodiments, the slopes of the two tip-axis goniometers have an identical absolute angle. In some embodiments, the absolute angle of the slope of one or each of the two tip-axis goniometers is about 3.6° (see FIG. 2 for an illustration). In some embodiments, the absolute angle of the slope of one or each of the two tip-axis goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, one or each of the two tip-axis goniometers is at or adjacent to a side surface (e.g., a vertical surface relative to the platform or the x-y motion stage) of the TnT motion platform. The two tip-axis goniometers can be at or adjacent to the same side surface or different side surfaces of the TnT motion platform. In some embodiments, one or each of the two tip-axis goniometers comprises a journal and a slanted pin. The material of the journal can comprise bronze. The material of the pin can comprise stainless steel. In some embodiments, one or each of the two tip-axis goniometers comprises a magnet. The magnet can retain contact between the journal and the slanted pin.

In some embodiments, the tilt-axis slanted linear bearing comprises a linear bearing carriage and a slanted linear bearing rail. In some embodiments, the linear bearing motion angle of the tilt-axis slanted linear bearing is about 3.4° (see FIGS. 3-4 for an illustration). In some embodiments, the linear bearing motion angle of the tilt-axis slanted linear bearing is, is about, is at least, is at least about, is at most, or is at most about, ±1°, ±1.1°, ±1.2°, ±1.3°, ±1.4°, ±1.5°, ±1.6°, ±1.7°, ±1.8°, ±1.9°, ±2°, ±2.1°, ±2.2°, ±2.3°, ±2.4°, ±2.5°, ±2.6°, ±2.7°, ±2.8°, ±2.9°, ±3.0°, ±3.1°, ±3.2°, ±3.3°, ±3.4°, ±3.5°, ±3.6°, ±3.7°, ±3.8°, ±3.9°, ±4°, ±4.1°, ±4.2°, ±4.3°, ±4.4°, ±4.5°, ±4.6°, ±4.7°, ±4.8°, ±4.9°, ±5°, ±5.1°, ±5.2°, ±5.3°, ±5.4°, ±5.5°, ±5.6°, ±5.7°, ±5.8°, ±5.9°, ±6°, ±6.1°, ±6.2°, ±6.3°, ±6.4°, ±6.5°, ±6.6°, ±6.7°, ±6.8°, ±6.9°, ±7°, ±7.1°, ±7.2°, ±7.3°, ±7.4°, ±7.5°, ±7.6°, ±7.7°, ±7.8°, ±7.9°, ±8°, ±8.1°, ±8.2°, ±8.3°, ±8.4°, ±8.5°, ±8.6°, ±8.7°, ±8.8°, ±8.9°, ±9°, ±9.1.°, ±9.2°, ±9.3°, ±9.4°, ±9.5°, ±9.6°, ±9.7°, ±9.8°, ±9.9°, ±10°, or a number or a range between any two of these values. In some embodiments, the absolute value of the linear bearing motion angle of the tilt-axis slanted linear bearing is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, the tilt-axis slanted linear bearing is at or adjacent a (or a second) side surface (e.g., a vertical surface relative to the platform or the x-y motion stage) of the TnT motion platform. In some embodiments, the TnT motion stage comprises a radial bearing in contact with a radial bearing rail. A material of the radial bearing can comprise stainless steel. A material of the radial bearing rail can comprise stainless steel. The radial bearing rail can be co-planar with the x-axis. In some embodiments, the TnT motion stage comprises at least one magnet (e.g., 2 magnets) which retains contact between the radial bearing and the radial bearing rail. In some embodiments, the radial bearing is at or adjacent to a side surface (or the second side surface). In some embodiments, the TnT motion stage comprises 8 side surfaces. In some embodiments, the TnT motion stage comprises 3, 4, 5, 6, 7, 8, 9, 10, 11, 12, 13, 14, 15, or a number or a range between any two of these values, side surfaces.

In some embodiments, when the tip-axis adjustment pawl is engaged with the tip-axis adjustment notch, the tilt-axis adjustment pawl is not engaged with the tilt-axis adjustment notch. When the tilt-axis adjustment pawl is engaged with the tilt-axis adjustment notch, the tip-axis adjustment pawl may be not engaged with the tip-axis adjustment notch. In some embodiments, when the tip-axis adjustment pawl is engaged with the tip-axis adjustment notch, a movement of the x-y motion stage along the x-axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the x-axis results in a movement of the TnT motion stage along the tilt-axis slanted linear bearing. This can result in a change in the tilt of the TnT motion stage.

In some embodiments, when the TnT motion stage is moved along the y-axis to the edge of its travel in one direction of the y-axis, the tip-axis adjustment pawl engages with the tip-axis adjustment notch. When the TnT motion stage is moved along the y-axis to the edge of its travel in the other direction of the y-axis, the tilt-axis adjustment pawl can engage with the tilt-axis adjustment notch.

Disclosed herein include embodiments of an instrument. In some embodiments, the instrument can comprise: a sensor (e.g., below or above the motion platform). The instrument can comprise: optics (e.g., below or above the motion platform). The instrument can comprise: a motion platform disclosed herein. The instrument can comprise a fluorescent imaging system, such as an optical genome mapping (OGM) system. In some embodiments, the base of the motion platform is fixed in position within the motion platform. In some embodiments, the motion platform is suspended within the instrument.

Disclosed herein include embodiments of a method of positioning a sample (e.g., adjusting the tip-axis and the tilt axis of the sample or TnT motion stage). In some embodiments, a method of positioning a sample can comprise: providing a sample. The method can comprise: placing the sample on (or onto or into) a sample carrier of a TnT motion stage of a motion platform of the present disclosure. The method can comprise: engaging the tip-axis adjustment paw with the tip-axis adjustment notch. The method can comprise: moving the x-y motion stage along one axis (e.g., the y-axis) of the x-axis and the y-axis. This can result in changing the tip of the TnT motion stage. The method can include: engaging the tilt-axis adjustment paw with the tilt-axis adjustment notch. The method can include: moving the x-y motion stage along the one axis (e.g., the y-axis) of the x-axis and the y-axis. This can result in changing the tilt of the TnT motion stage. Prior to engaging the tilt-axis adjustment paw with the tilt-axis adjustment notch, the method can include: disengaging the tip-axis adjustment paw with the tip-axis adjustment notch

In some embodiments, engaging the tip-axis adjustment paw with the tip-axis adjustment notch and moving the x-y motion stage along the one axis of the x-axis and the y-axis occurs before engaging the tilt-axis adjustment paw with the tilt-axis adjustment notch and moving the x-y motion stage along the one axis of the x-axis and the y-axis, thereby changing the tilt of the TnT motion stage. Prior to engaging the tilt-axis adjustment paw with the tilt-axis adjustment notch, the method can include: disengaging the tip-axis adjustment paw with the tip-axis adjustment notch. In some embodiments, engaging the tip-axis adjustment paw with the tip-axis adjustment notch and moving the x-y motion stage along the one axis of the x-axis and the y-axis occurs after engaging the tilt-axis adjustment paw with the tilt-axis adjustment notch and moving the x-y motion stage along the one axis of the x-axis and the y-axis, thereby changing the tilt of the TnT motion stage. Prior to engaging the tip-axis adjustment paw with the tip-axis adjustment notch, the method can include: disengaging the tilt-axis adjustment paw with the tilt-axis adjustment notch. In some embodiments, the method comprises: changing the x-y position of the motion stage. Changing the x-y position of the motion stage can occur before or after changing the tip of the TnT motion stage and/or the tilt of the TnT motion stage.

Disclosed herein include methods of imaging a sample. In some embodiments, a method of imaging a sample comprising: positioning (e.g., adjusting the tip-axis and/or tilt-axis) a sample as described herein. The sample can be on (or in) a sample carrier of a TnT motion stage of a motion platform of the present disclosure. Positing the sample can include adjusting the tip-axis and/or tilt-axis of the TnT motion stage as described herein. The method can include: rastering, using the x-y motion stage, to different positions along the x-axis and/or the y-axis. The method can include capturing images of the sample at the different positions. In some embodiments, the tip-axis and/or tilt-axis of the sample (or the TnT motion stage) can be adjusted after a number of images of the sample are captured at different positions. In some embodiments, the tip-axis and/or tilt-axis of the sample (or the TnT motion stage) may not need to be adjusted.

In some embodiments, the sample comprises an optical genome mapping (OGM) sample. In some embodiments, the sample comprises nucleic acids. The nucleic acids can comprise deoxyribonucleic acid (DNA). The nucleic acids can comprise the nucleic acids comprise genomic DNA. The nucleic acids can comprise fragmented genomic DNA. The nucleic acids can comprise ribonucleic acids (RNA). The nucleic acids can comprise DNA derived (e.g., reverse transcribed) from DNA or RNA. In some embodiments, the sample comprises labeled nucleic acids, optionally wherein the sample comprises fluorescently labeled nucleic acids.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates a non-limiting exemplary illustration of a motion platform.

FIG. 2 shows a non-limiting exemplary illustration of components of a motion platform for adjusting an axis (a tip axis illustrated) of a motion stage (e.g., a tip and tilt motion stage).

FIG. 3 shows a non-limiting exemplary illustration of components of a motion platform for adjusting an axis (a tilt axis illustrated) of a motion stage (e.g., a tip and tilt motion stage).

FIG. 4 shows a non-limiting exemplary illustration of adjusting an axis (a tilt axis illustrated) of a motion stage (e.g., a tip and tilt motion stage).

FIGS. 5A-5Z and 5AA-5AD are frames of a video showing a non-limiting exemplary adjustment of an axis (a tip axis illustrated) of a motion stage (e.g., a tip and tilt motion stage) followed by a non-limiting exemplary adjustment of another axis (a tilt axis illustrated) of the motion stage. The design presented herein can move the Y axis to the edge of its travel where it engages with a pawl (FIGS. 5A-5B; see FIGS. 1-2) which permits the sample carrier to be moved along two inclined journals (goniometers) that can affect the tip axis (FIGS. 5C-5K; see FIGS. 1-2). The design thereafter can move the Y axis to the opposite end where a different pawl engages a slanted bearing (FIGS. 5L-5Y; see FIGS. 3-4). Once the secondary pawl is engaged with the slanted bearing, motion in the X-axis affects an effective tilt motion (FIGS. 5Z-5AB; see FIGS. 3-4). This novel design enables four axes (tip, tilt, x, y) to be affected by only two motors rather than four, and the motors are mounted stationary, rather than on the TnT axis, thereby further reducing the moving-mass.

FIG. 6. Exemplary gradient planes and angles definitions.

FIG. 7A. Exemplary leveling measurement during alignment measures chip gradient φC.

FIG. 7B. Exemplary image Z stack measures the difference between the chip plane and the FOV focal plane, φ_{L}.

FIG. 8. Exemplary hardware configuration parameters.

FIG. 9. Exemplary planar transform.

FIG. 10. Exemplary internal X, Z coordinate system.

FIG. 11. Exemplary parametric t calculation.

FIG. 12. Exemplary plate correction.

FIG. 13. Exemplary q approximation.

FIG. 14. Exemplary stage X offset to apply X component of gradient.

FIG. 15. Exemplary rotational shift x and z components.

FIG. 16. Exemplary plate vector calculations.

FIG. 17. Exemplary calibrating gonio slopes. Residuals added until desired slope of 1 achieved.

FIG. 18 is a block diagram of an illustrative computing system configured to implement tip and tilt motion stage control.

FIG. 19 illustrates a non-limiting exemplary workflow of OGM.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

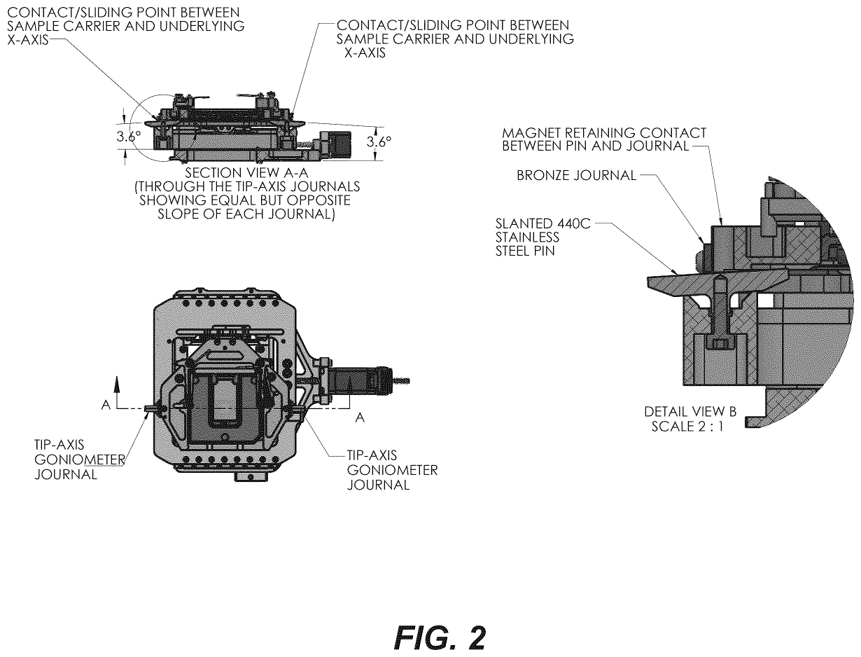

Disclosed herein includes embodiments of a motion platform. In some embodiments, a motion platform can comprise: a base. The motion platform can comprise: an x-y motion stage. The x-y motion stage can be on (e.g., attached to, such as securely attached to) the base. The motion platform can comprise: a tip-axis adjustment pawl (or a tip-axis adjustment engagement component) on the base. The motion platform can comprise: a tilt-axis adjustment pawl (or a tilt-axis adjustment engagement component) on (e.g., attached to, such as securely attached to) the base. The motion platform can comprise: a tip and tilt (TnT) motion stage. The TnT motion stage can be on the x-y motion stage. The TnT motion stage can comprise: two tip-axis goniometers with different slopes (e.g., 3.6° and −3.6° respectively as illustrated in FIG. 2) relative to one axis (e.g., x-axis) of the x-axis and the y-axis (e.g., of the motion platform or the motion stage). The TnT motion stage can comprise: a bearing, such as a tilt-axis slanted linear bearing (e.g., slanted relative to the plane of the platform and/or the x-y motion stage). The bearing can be a recirculating linear bearing. The bearing can be a non-recirculating linear bearing. The TnT motion stage can comprise: a tip-axis adjustment notch (or a tip-axis complementary adjustment engagement component). The TnT motion stage can comprise: a tilt-axis adjustment notch (or a tilt-axis complementary adjustment engagement component). In some embodiments, the TnT motion stage can comprise: a sample carrier. In some embodiments, when the tip-axis adjustment pawl is engaged with the tip-axis adjustment notch, a movement of the x-y motion stage along the one axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

Disclosed herein include embodiments of a motion platform. In some embodiments, a motion platform can comprise: a base. The motion platform can comprise: an x-y motion stage on the base. The motion platform can comprise: a tip-axis adjustment pawl (or a tip-axis adjustment engagement component) on the base. The motion platform can comprise: a tip and tilt (TnT) motion stage. The TnT motion stage can be on the x-y motion stage. The TnT motion stage can comprise: one or more (e.g., 2) tip-axis goniometers with different slopes relative to one axis of the x-axis and the y-axis. The TnT motion stage can comprise: a tip-axis adjustment notch. In some embodiments, when the tip-axis adjustment pawl is engaged with the tip-axis adjustment notch, a movement of the x-y motion stage along the one axis results in a movement of the TnT motion stage along the one or more tip-axis goniometers. This can result in a change in the tip of the TnT motion stage. In some embodiments, the TnT motion stage can comprise: a sample carrier.

Disclosed herein include embodiments of a motion platform. In some embodiments, a motion platform can comprise: a base. The motion platform can comprise: an x-y motion stage on the base. The motion platform can comprise: a tilt-axis adjustment pawl (or a tilt-axis adjustment engagement component) on the base. The motion platform can comprise: a tip and tilt (TnT) motion stage. The TnT motion stage can be on the x-y motion stage. The TnT motion stage can comprise: a tilt-axis slanted linear bearing. The TnT motion stage can comprise: a tilt-axis adjustment notch (or a tilt-axis complementary adjustment engagement component). In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage. In some embodiments, the TnT motion stage can comprise: a sample carrier.

Disclosed herein include embodiments of a motion platform. In some embodiments, a motion platform can comprise: a base. The motion platform can comprise: an x-y motion stage. The x-y motion stage can be on (e.g., attached to, such as securely attached to) the base. The motion platform can comprise: a first-axis (e.g., a tip-axis or a tilt-axis) adjustment engagement component (e.g., a pawl or a notch) on (e.g., attached to, such as securely attached to) the base. The motion platform can comprise: a tip and tilt (TnT) motion stage. The TnT motion stage can be on the x-y motion stage. The motion platform can comprise: a first-axis goniometer or bearing. The motion platform can comprise: a first-axis complementary adjustment engagement component (e.g., a notch or a pawl), The first-axis adjustment engagement component and the first-axis complementary adjustment engagement component can engage (or be in engagement, such as secure engagement) with each other. For example, the first-axis adjustment engagement component and the first-axis complementary engagement component can be a pawl and a notch and can be in engagement (e.g., secure engagement) with each other. When the first-axis adjustment component is engaged with the first-axis complementary adjustment engagement component, a movement of the x-y motion stage along one axis (e.g., x-axis) of the x-axis and the y-axis can result. in a movement of the TnT motion stage along the first-axis goniometer or bearing. This can result in a change in the first-axis (e.g., tip-axis) of the TnT motion stage. In some embodiments, the motion platform can comprise: a sample carrier.

Disclosed herein include embodiments of an instrument. In some embodiments, the instrument can comprise: a sensor (e.g., below or above the motion platform). The instrument can comprise: optics (e.g., below or above the motion platform). The instrument can comprise: a motion platform disclosed herein. The instrument can comprise a fluorescent imaging system, such as an optical genome mapping (OGM) system. In some embodiments, the base of the motion platform is fixed in position within the motion platform. In some embodiments, the motion platform is suspended within the instrument.

Disclosed herein include embodiments of a method of positioning a sample (e.g., adjusting the tip-axis and the tilt axis of the sample or TnT motion stage). In some embodiments, a method of positioning a sample can comprise: providing a sample. The method can comprise: placing the sample on (or onto or into) a sample carrier of a TnT motion stage of a motion platform of the present disclosure. The method can comprise: engaging the tip-axis adjustment paw with the tip-axis adjustment notch. The method can comprise: moving the x-y motion stage along one axis (e.g., the y-axis) of the x-axis and the y-axis. This can result in changing the tip of the TnT motion stage. The method can include: engaging the tilt-axis adjustment paw with the tilt-axis adjustment notch. The method can include: moving the x-y motion stage along the one axis (e.g., the y-axis) of the x-axis and the y-axis. This can result in changing the tilt of the TnT motion stage. Prior to engaging the tilt-axis adjustment paw with the tilt-axis adjustment notch, the method can include: disengaging the tip-axis adjustment paw with the tip-axis adjustment notch

Disclosed herein include methods of imaging a sample. In some embodiments, a method of imaging a sample comprising: positioning (e.g., adjusting the tip-axis and/or tilt-axis) a sample as described herein. The sample can be on (or in) a sample carrier of a TnT motion stage of a motion platform of the present disclosure. Positing the sample can include adjusting the tip-axis and/or tilt-axis of the TnT motion stage as described herein. The method can include: rastering, using the x-y motion stage, to different positions along the x-axis and/or the y-axis. The method can include capturing images of the sample at the different positions. In some embodiments, the tip-axis and/or tilt-axis of the sample (or the TnT motion stage) can be adjusted after a number of images of the sample are captured at different positions. In some embodiments, the tip-axis and/or tilt-axis of the sample (or the TnT motion stage) may not need to be adjusted.

### Motion Platform Design

Microscopy instruments, such as fluorescent imaging instruments (e.g., optical genome mapping (OGM) instruments), can require high-magnification optics to produce images with sufficiently resolution of the molecules being imaged (e.g., DNA molecules being imaged). High-magnification optics can have a narrow depth-of-focus, meaning that the sample being imaged must be placed precisely at the appropriate distance from the optics to produce focused images. Throughput of instruments can be generally proportionate to the field of view (FoV) size of images acquired. Instrument architecture can therefore require the largest possible FOV size, however, at the cost of having to precisely position the sample at the precise distance from the optics. To position samples at the precise position relative to the optics, microscopy instruments can generally employ an XY motion stage (or XY stage), paired with a Tip and Tilt (TnT) motion stage (Or TnT stage). The XY motion stage (or XY stage) is also referred to herein as an x-y motion stage (or x-y stage). The XY motion stage can move the center of the FoV to the appropriate location of the sample to be imaged, while the TnT stage can pivot to an appropriate plane to ensure that the FoV (e.g., the complete or entire FoV or a sufficiently large FoV) is sufficiently perpendicular to the optical axis. Images acquired without the appropriate TnT adjustment can produce images with only a portion (e.g., a linear portion) of the image being in focus rather than the entire FoV (or a sufficient large FoV) being in focus.

A lot of (e.g., most of) fluorescence microscopy instruments raster scans many successive images. Thus, minimizing move times between FoVs to maximize instrument throughput can be advantageous. To minimize move times, the moving mass can be kept to a minimum. Motorized TnT motion mechanisms are generally heavy. To minimize moving mass, microscopy instruments can therefore be architected with the XY motion stage on top of the TnT motion stage. This design has historically produced the lowest moving-mass design. However this design can introduce a fundamental constraint.

The dynamic nature of TnT stages means that they have poor structural stiffness (which can be almost by design). By mounting the XY stage on top of a TnT stage, a lightweight design is achieved; however the XY motion (also referred to herein as x-y motion) can impart a shockwave impulse into the structure that vibrationally perturbs it. A lengthy ringdown period after the XY move (also referred to herein as x-y move) can be required to dissipate the energy before image acquisition can start. Initiating image acquisition before resonance has been attenuated can produce blurry images. Fluorescent imaging, such as OGM imaging, can require attenuation of resonance to less than, for example, 40 nm before imaging may commence. For reference, this is approximately 1/10^{th} the wavelength of blue light, and can be exceptionally challenging to achieve consistently.

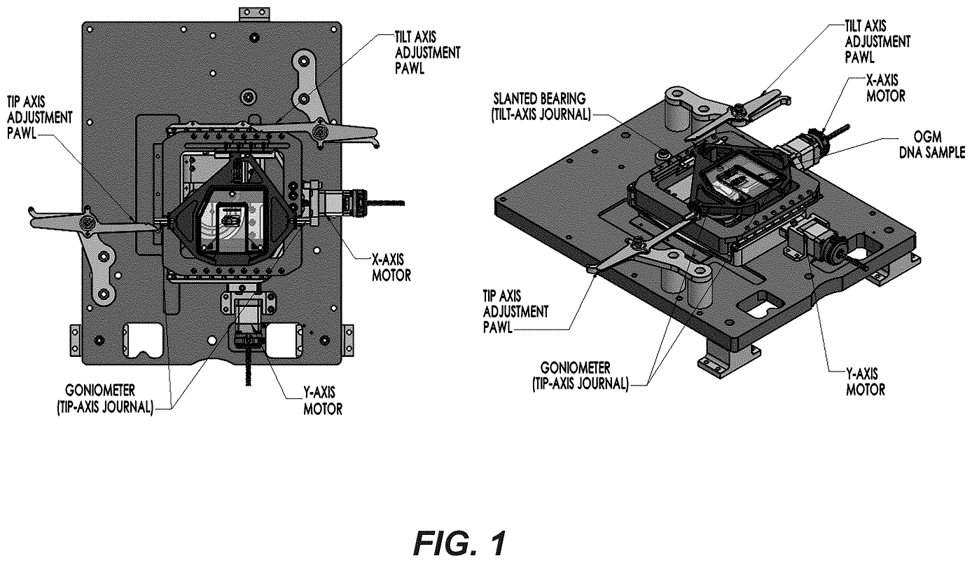

An alternative design (or architecture) is disclosed herein with the mass of the TnT stage being substantially reduced to a point where it can be mounted on top of the XY stage, rather than beneath it (see FIG. 1 for an illustration). This architecture presents a mass lighter (nimbler) than legacy designs and can avert the fundamental flaw of mounting an XY axis on a TnT stage which inherently compromises structural stiffness.

Legacy TnT stages employ a servo-controlled motor for each of the tip and tilt axes. Each of these axes will furthermore require journals or bearings to allow the motion of each axis. The motors can typically constitute the majority of the mass that renders legacy designs too heavy to be mounted on top of XY stages. The novel design disclosed herein can minimize (e.g., completely omit) the motors that drive the tip and tilt axes. The design being presented can utilize the underlying XY motors to adjust the TnT axes before commencing with raster scanning. Legacy systems would control the four servo motors that control X, Y, tip, and tilt independently.

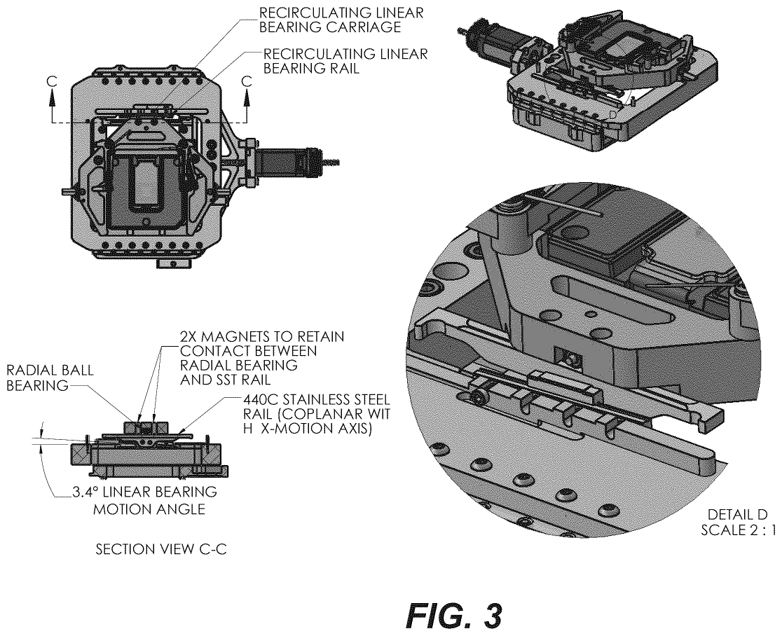

In some embodiments, the design presented herein can move the Y axis to the edge of its travel where it engages with a pawl (see FIGS. 1-2 and FIGS. 5A-5B for illustrations) which permits the sample carrier to be moved along two inclined journals (goniometers) that can affect the tip axis (see FIGS. 1-2 and FIGS. 5C-5K for illustrations). The system thereafter can move to the opposite end of the Y axis where a different pawl engages a slanted bearing (see FIGS. 3-4 and FIGS. 5L-5Y for illustrations). Once the secondary pawl is engaged with the slanted bearing, motion in the X-axis affects an effective tilt motion (see FIGS. 3-4 and FIGS. 5Z-5AB for illustrations). This novel design enables four axes (tip, tilt, x, y) to be affected by only two motors rather than four, and the motors are mounted stationary, rather than on the TnT axis, thereby further reducing the moving-mass.

Exemplary use. In some embodiments, designs disclosed herein can offer utility in high magnification microscopy applications (e.g., all high magnification microscopy applications) that employ relatively large FoVs and high numerical aperture (NA) optics.

Exemplary improvements and advantages. In some embodiments, motion times can be significantly reduced with the designs disclosed herein. For example, the move times between successive FoVs can be significantly reduced compared to legacy (or prior) designs. Prior designs can generally require ˜210 milliseconds for an XY move (with ringdown), whereas the design disclosed herein can achieve an XY move in ˜90 milliseconds. Such reduction can increase system throughput. In some embodiments, initial instrument cost (COGS) can be reduced (e.g., significantly reduced) since two motors perform the tasks previously requiring four motors. In some embodiments, maintenance cost can be reduced since there are fewer motors to maintain. In some embodiments, a design of the present disclosure can have a smaller physical size (more compact). In some embodiments, software required to manipulate and navigate the geometric space can be simplified and easier to develop.

Ringdown period. Long ringdown periods (e.g., about 130 milliseconds) can be reduced (e.g., to about 20 milliseconds) on the platforms disclosed herein. In some embodiments, the ringdown time is, is about, is at least, is at least about, is at most, or is at most about, 5 milliseconds (ms), 6 ms, 7 ms, 8 ms, 9 ms, 10 ms, 11 ms, 12 ms, 13 ms, 14 ms, 15 ms, 16 ms, 17 ms, 18 ms, 19 ms, 20 ms, 21 ms, 22 ms, 23 ms, 24 m, 25 ms, 26 ms, 27 ms, 28 ms, 29 ms, 30 ms, 35, ms, 40 ms, 45 ms, 50 ms, 55 ms, 60 ms, 65 ms, 70 ms, 75 ms, 80 ms, 85 ms, 90 ms, 95 ms, 100 ms, 105 ms, 110 ms, or a number or a range between any two of these values.

Motion times. The benefit can be recognized in the difference of ringdown duration. A typical move consists of a 70 millisecond move time, followed by either a 20 millisecond ringdown period (for the systems and designs disclosed herein) versus a 130 millisecond ringdown for the legacy designs. This equates to about 210 millisecond move (including ringdown time) for the legacy system compared to only 90 ms for the systems and designs disclosed herein. In some embodiments, the motion time of designs, systems, platforms, and methods of the present disclosure is, is about, is at least, is at least about, is at most, or is at most about, 70 milliseconds (ms), 75 ms, 80 ms, 85 ms, 90 ms, 95 ms, 100 ms, 105 ms, 110 ms, 115 ms, 120 ms, 125 ms, 130 ms, 135 ms, 140 ms, 145 ms, 150 ms, 155 ms, 160 ms, 165 ms, 170 ms, 175 ms, 180 ms, or a number or a range between any two of these values.

Throughput. A design of the present disclosure can have a throughput improvement (relative to the throughput of a prior design) of, of about, of at least, of at least about, of at most, or of at most about, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, or a number or a range between any two of these values.

Materials. In some embodiments, a component (e.g., a bearing) of the a design disclosed herein can be made from alloys (e.g., aluminum alloys). In some embodiments, a component (e.g., a bearing) of the design herein can be fabricated from a steel (e.g., stainless steel). In some embodiments, an alloy can comprise aluminum alloys, zinc alloys, copper alloys, titanium alloys, tin alloys, beryllium alloys, bismuth alloys, chromium alloys, cobalt alloys, gallium alloys, indium alloys, iron alloys, manganese alloys, nickel alloys, rhodium alloys, or a combination thereof. In some embodiments, a component (e.g., a bearing) of the design herein can be fabricated from a steel, such as cold rolled steel, stainless steel and steel surface-treated steel. In some embodiments, a steel can be crucible steel, carbon steel, spring steel, alloy steel, maraging steel, stainless steel, high-speed steel, weathering steel, tool steel, or a combination thereof. In some embodiments, a component of the design herein can comprise brass.

Weights. The legacy designs would include two motors for the TnT functionality along with their respective supports and bearings. The collective mass for those would generally be greater than 700 grams. The presently disclosed mechanism's net weight to achieve the TnT functionality can be less than 150 grams, for example. In some embodiments, the presently disclosed mechanism's net weight to achieve the TnT functionality can be, be about, be at least, be at least about, be at most, or be at most about, 100 grams (g), 110 g, 120 g, 130 g, 140 g, 150 g, 160 g, 170 g, 180 g, 190 g, 200 g, 225 g, 250 g, 275 g, 300 g, 325 g, 350 g, 375 g, 400 g, 425 g, 450 g, 475 g, 500 g, or a number or a range between any two of these values.

Size. The design disclosed herein can result in, for example, at least a 50% reduction in physical volume consumed by the mechanisms compared to prior designs. In some embodiments, the reduction in physical volume of a design disclosed herein can be, be about, be at least, be at least about, be at most, or be at most about, 80%, 75%, 70%, 65%, 60%, 55%, 50%, 45%, 40%, 35%, 30%, 25%, or a number or a range between any two of these values.

### Exemplary Motion Platform

Disclosed herein includes embodiments of a motion platform. Referring to FIG. 1, a motion platform can comprise: a base. A dimension (e.g., length or depth) of a component of a motion platform, e.g., a base, or a component or a base, can be, be about, be at least, be at least about, be at most, or be at most about, 10 cm, 15 cm, 20 cm, 25 cm, 30 cm, 40 cm, 50 cm, 60 cm, 70 cm, 80 cm, 90 cm, 100 cm, or a number or a range between any two of these values. An area of a component of a motion platform, e.g., a base (such as top surface area where the x-y motion stage is mounted), or a component or a base of the component of the motion platform or the component of the base can be, be about, be at least, be at least about, be at most, or be at most about, 100 cm², 150 cm², 200 cm², 250 cm², 300 cm², 400 cm², 500 cm², 600 cm², 700 cm², 800 cm², 900 cm², 1000 cm², 2500 cm², 5000 cm², 7500 cm², 10000 cm², or a number or a range between any two of these values.

The motion platform can comprise: an x-y motion stage. A dimension (e.g., length or depth) of a component of a motion platform, e.g., an x-y motion stage, or a component of an x-y motion stage, can be, be about, be at least, be at least about, be at most, or be at most about, 5 cm, 6, cm, 7 cm, 8 cm, 9 am, 10 cm, 15 cm, 20 cm, 25 cm, 30 cm, 40 cm, 50 cm, 60 cm, or a number or a range between any two of these values. An area of a component of a motion platform, e.g., an x-y motion stage (such as the top surface area wherein the TnT motion stage is on), or a component of an x-y motion stage can be, be about, be at least, be at least about, be at most, or be at most about, 100 cm², 150 cm², 200 cm², 250 cm², 300 cm², 400 cm², 500 cm², 600 cm², 700 cm², 800 cm², 900 cm², 1000 cm², 2500 cm², 3000 cm², 400 cm², 5000 cm², or a number or a range between any two of these values. The x-y motion stage can be on (e.g., attached to, such as securely attached to) the base.

The motion platform can comprise: a tip-axis adjustment pawl (or a tip-axis adjustment engagement component) on the base. The motion platform can comprise: a tilt-axis adjustment pawl (or a tilt-axis adjustment engagement component) on (e.g., attached to, such as securely attached to) the base. A dimension (e.g., length or depth) of a tip-axis adjustment engagement component or tilt-axis adjustment engagement component can be, be about, be at least, be at least about, be at most, or be at most about, 2 cm, 3 cm, 4 cm, 5 cm, 6 cm, 7 cm, 8 cm, 9 cm, 10 cm, 11 cm, 12 cm, 13 cm, 14 cm, 15 cm, or a number or a range between any two of these values. The motion platform can comprise: a tip and tilt (TnT) motion stage. The TnT motion stage can be on the x-y motion stage. A dimension (e.g., width or length) of a component of the motion platform or a component of the TnT motion stage, e.g., a tip-axis adjustment engagement component, a tilt-axis adjustment engagement component, a journal, a slanted pin, a linear bearing carriage, a slanted linear bearing rail, or a sample carrier, can be, be about, be at least, be at least about, be at most, or be at most about, 3 cm, 4 cm, 5 cm, 6, cm, 7 cm, 8 cm, 9 am, 10 cm, 15 cm, 20 cm, 25 cm, 30 cm, 40 cm, or a number or a range between any two of these values. An area of a component of the motion platform or a component of the TnT motion stage, e.g., a tip-axis adjustment engagement component, a tilt-axis adjustment engagement component, a journal, a slanted pin, a linear bearing carriage, a slanted linear bearing rail, or a sample carrier, can be, be about, be at least, be at least about, be at most, or be at most about, 10 cm², 20 cm², 30 cm², 40 cm², 50 cm², 75 cm², 100 cm², 150 cm², 200 cm², 250 cm², 300 cm², 400 cm², 500 cm², 600 cm², 700 cm², 800 cm², 900 cm², 1000 cm², 1500 cm², 2000 cm², or a number or a range between any two of these values.

Referring to FIGS. 1-2, the TnT motion stage can comprise: two tip-axis goniometers with different slopes (e.g., 3.6° and −3.6° respectively as illustrated in FIG. 2) relative to one axis (e.g., x-axis) of the x-axis and the y-axis (e.g., of the motion platform or the motion stage). The TnT motion stage can comprise: a bearing, such as a tilt-axis slanted linear bearing (e.g., slanted relative to the plane of the platform and/or the x-y motion stage). The bearing can be a recirculating linear bearing. The bearing can be a non-recirculating linear bearing.

Referring to FIGS. 1 and 3-4, the TnT motion stage can comprise: a tip-axis adjustment notch (or a tip-axis complementary adjustment engagement component). The TnT motion stage can comprise: a tilt-axis adjustment notch (or a tilt-axis complementary adjustment engagement component). In some embodiments, the TnT motion stage can comprise: a sample carrier.

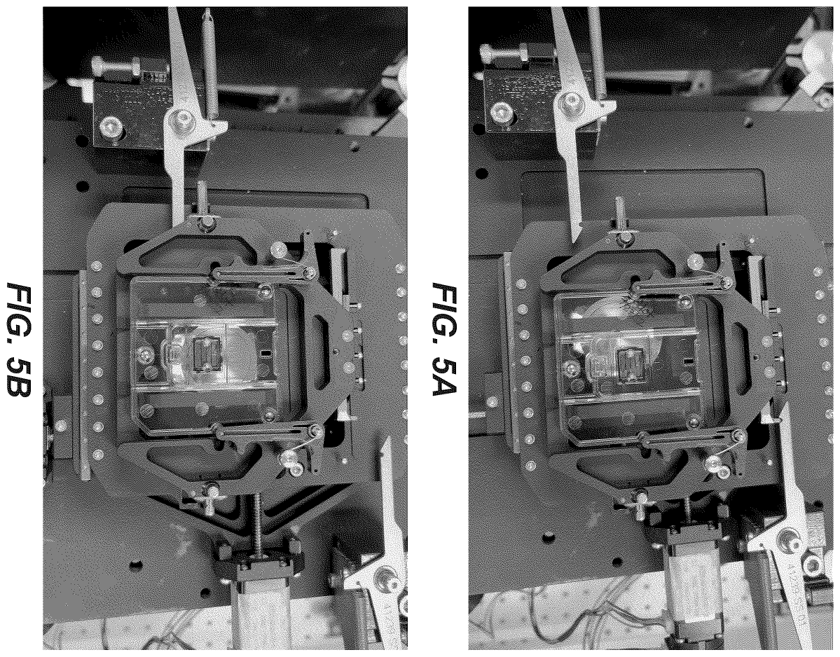

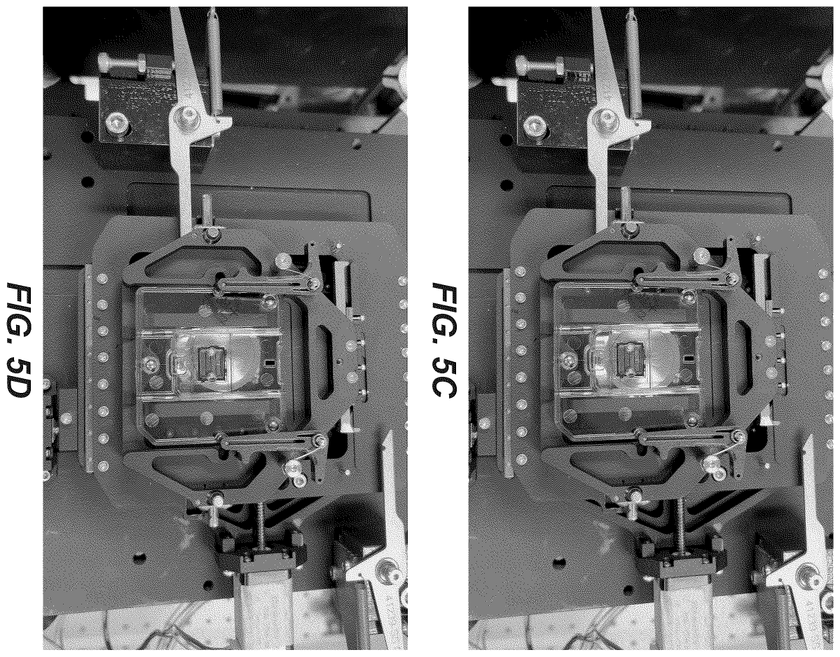

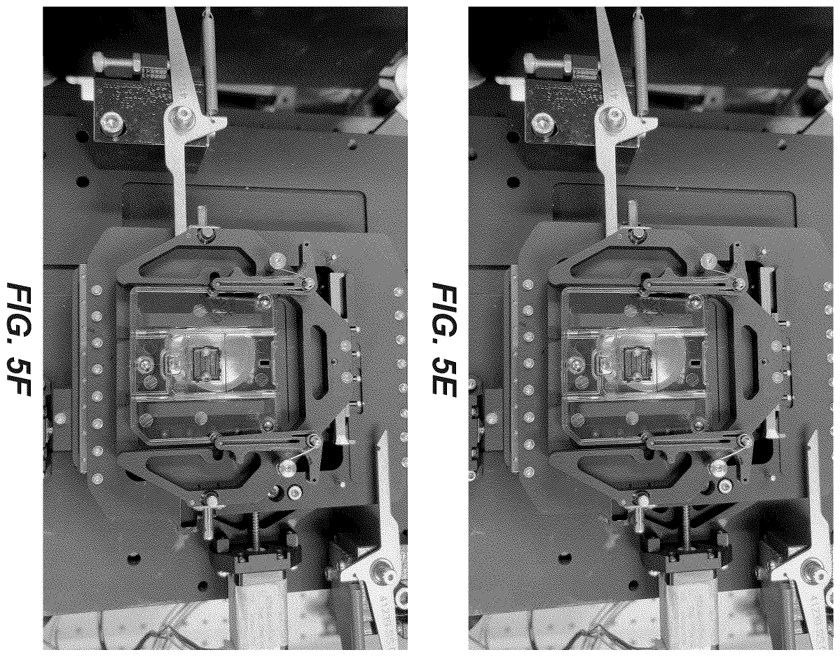

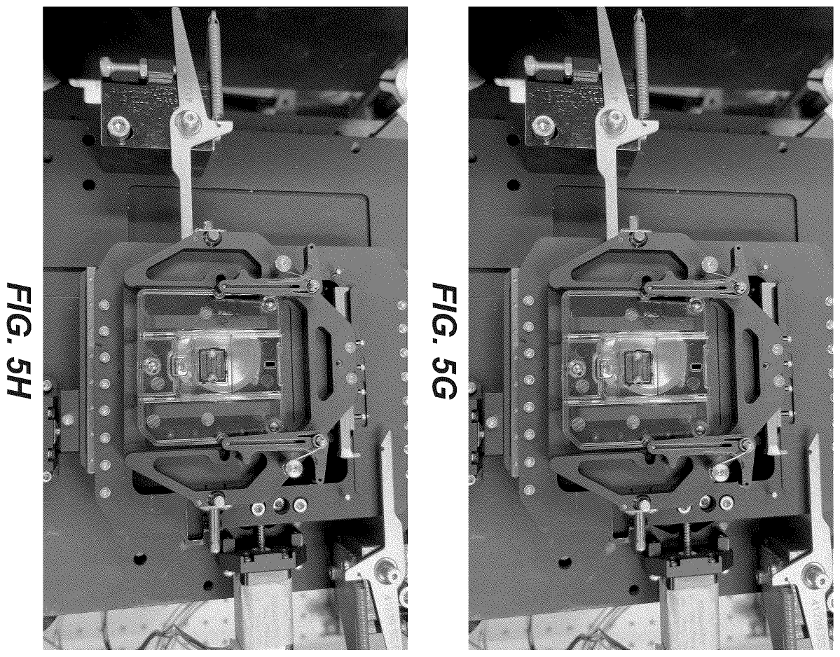

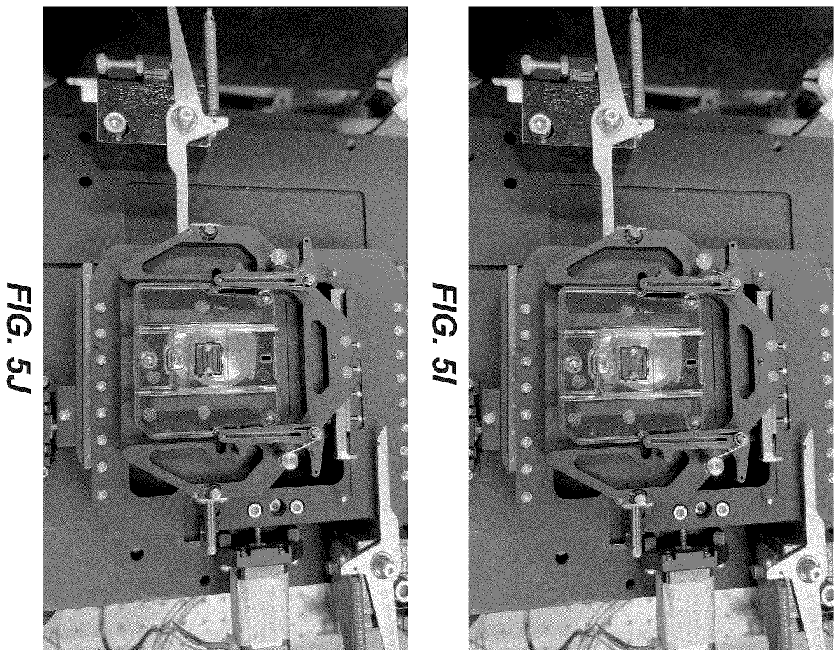

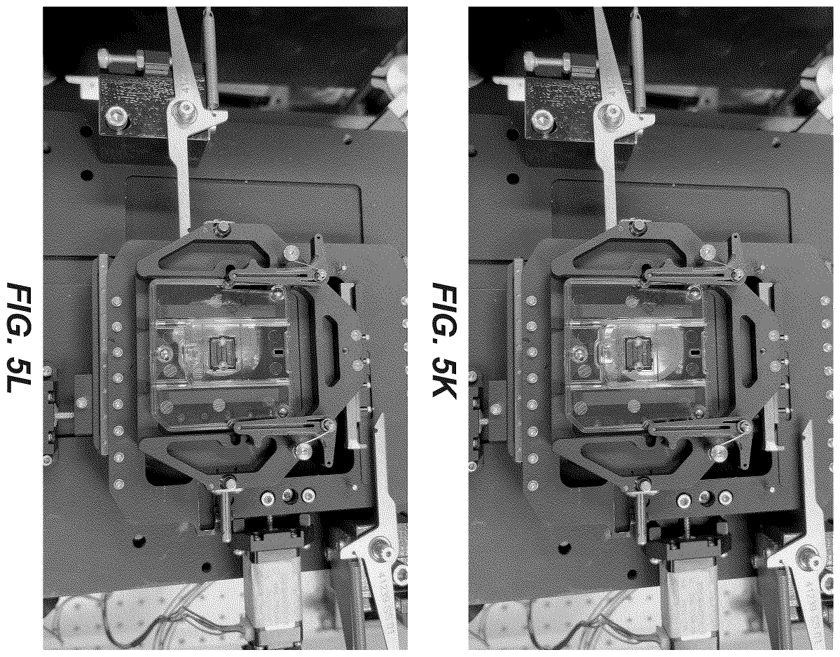

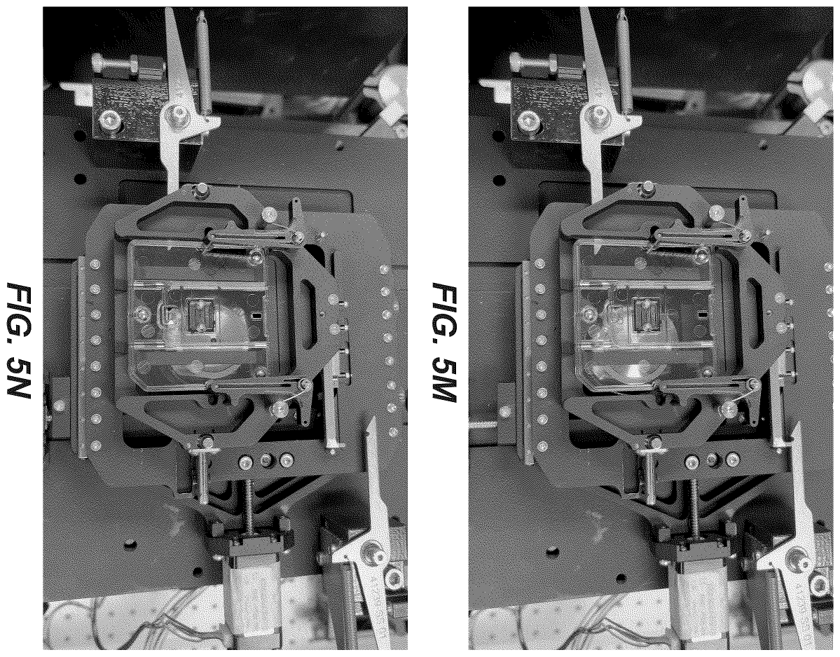

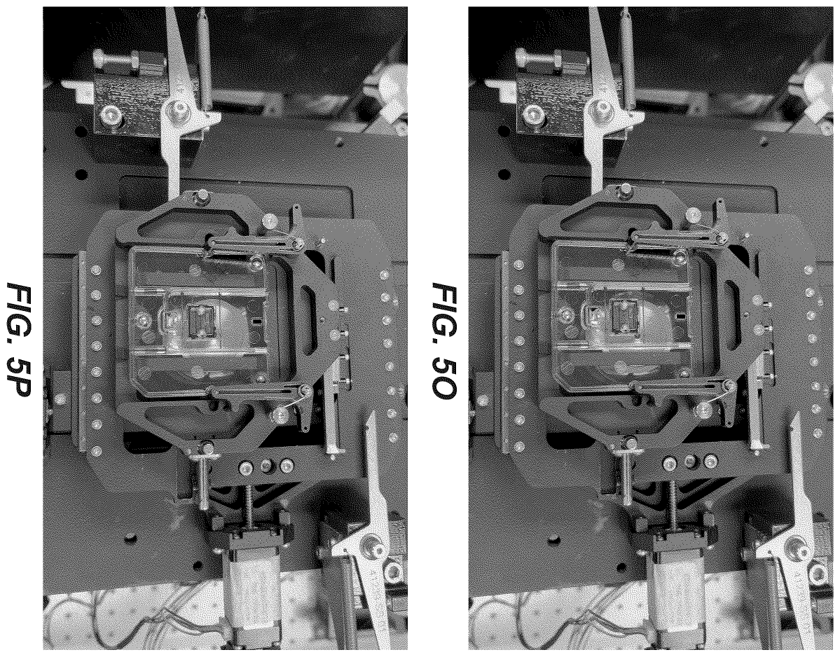

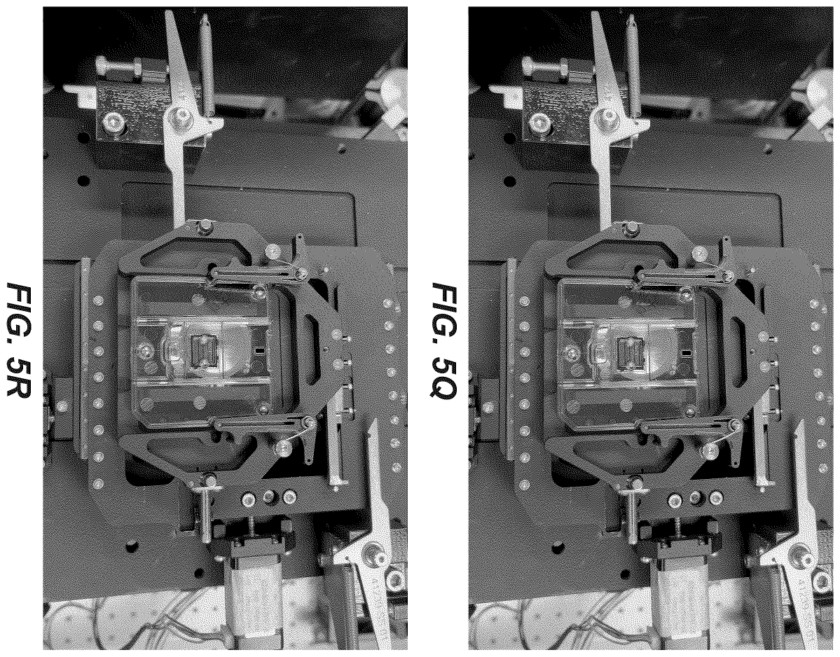

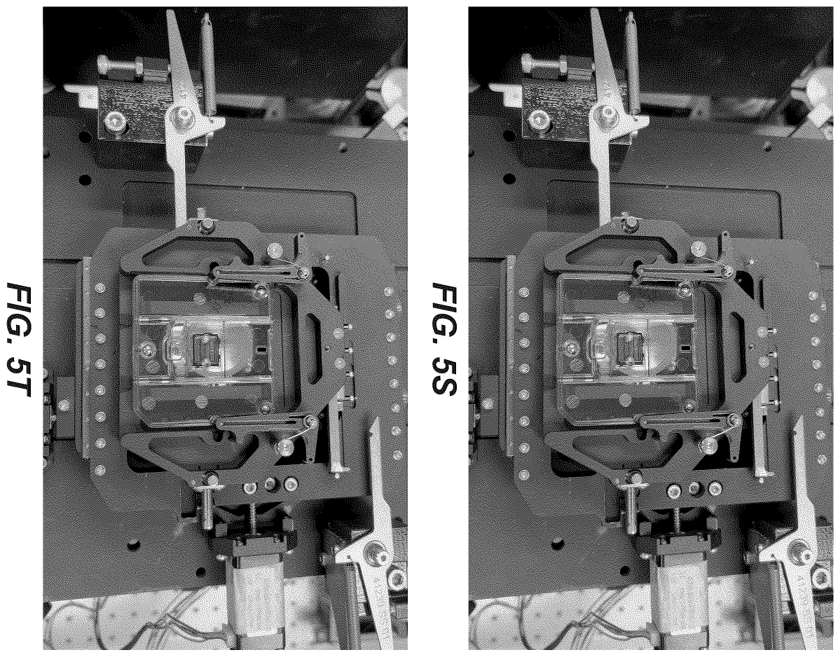

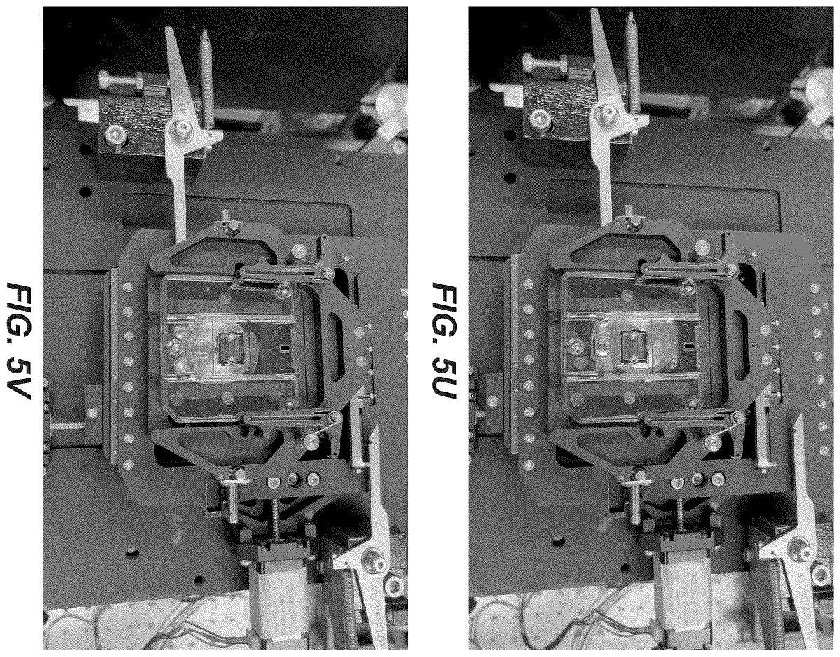

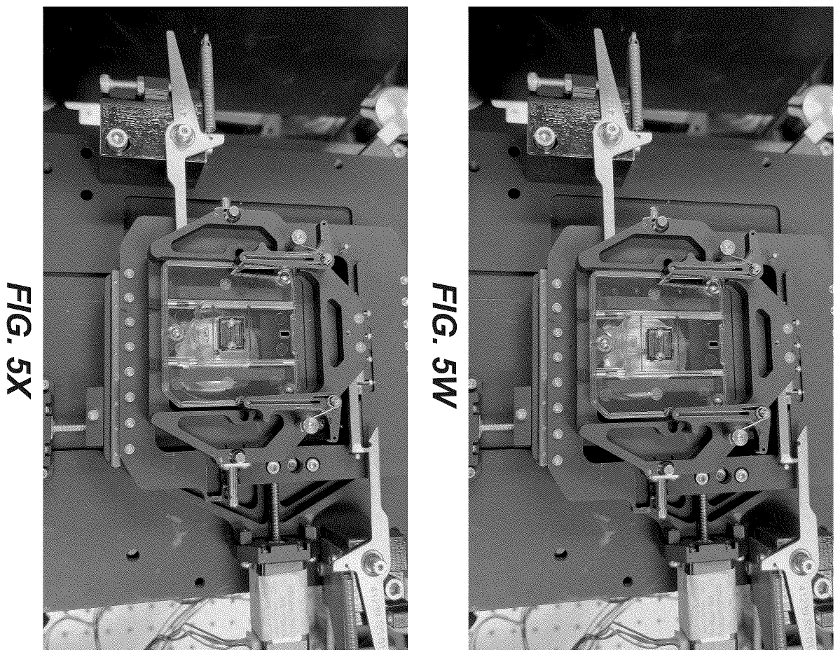

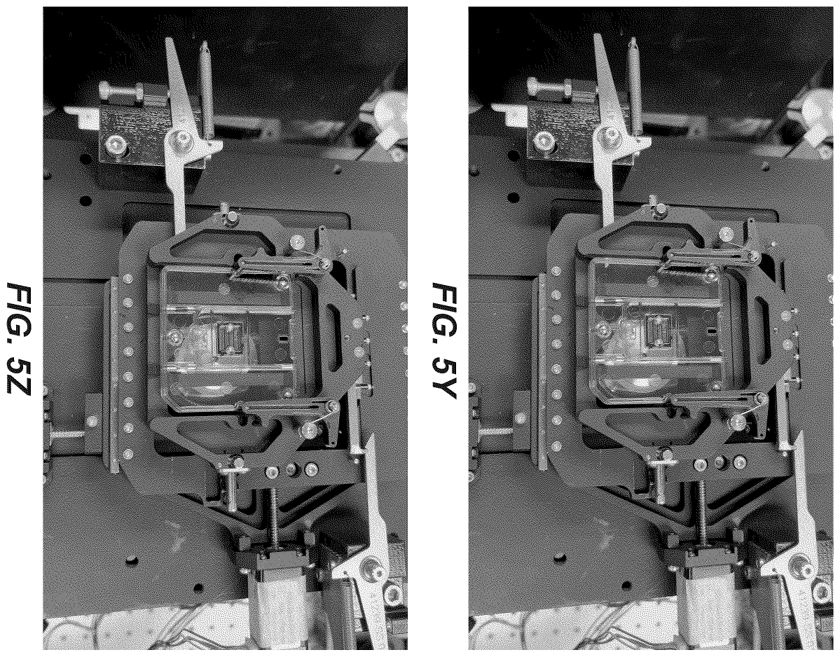

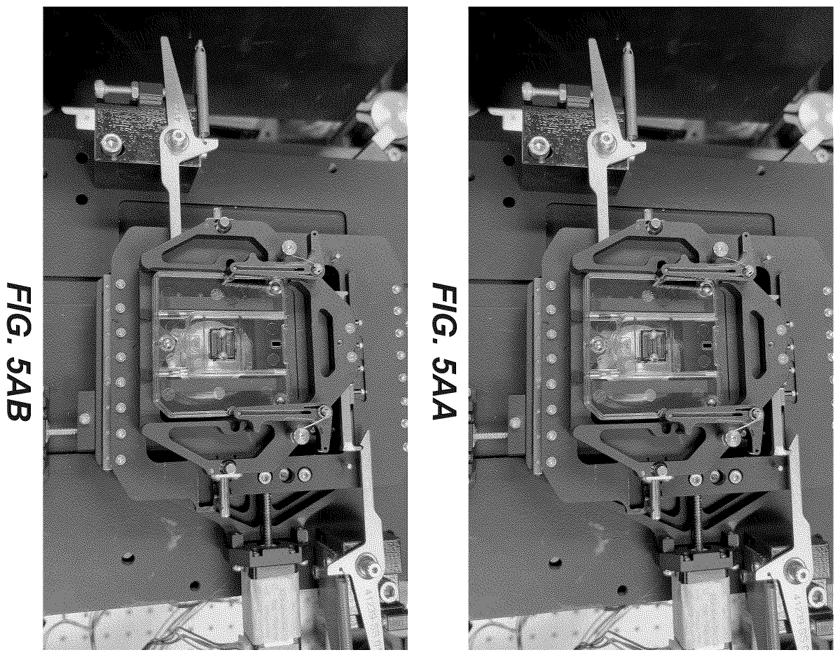

Referring to FIGS. 5A-5AD, in some embodiments, when the tip-axis adjustment pawl is engaged with the tip-axis adjustment notch, a movement of the x-y motion stage along the one axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

In some embodiments, a motion platform can comprise: a base. The motion platform can comprise: an x-y motion stage on the base. The motion platform can comprise: a tip-axis adjustment pawl (or a tip-axis adjustment engagement component) on the base. The motion platform can comprise: a motion stage. The tip motion stage can be on the x-y motion stage.

The Tip motion stage can comprise: one or more (e.g., 2, or 3, 4, 5, or more) tip-axis goniometers with different (or the same) slopes relative to one axis of the x-axis and the y-axis. The TnT motion stage can comprise: a tip-axis adjustment notch. When the tip-axis adjustment pawl is engaged with the tip-axis adjustment notch, a movement of the x-y motion stage along the one axis can result in a movement of the tip motion stage along the one or more tip-axis goniometers. This can result in a change in the tip of the tip motion stage. In some embodiments, the tip motion stage can comprise: a sample carrier.

The motion platform can further comprise: a tilt-axis adjustment pawl (or a tilt-axis adjustment engagement component) on the base. The tip motion stage can be a tip and tilt (TnT) motion stage. The TnT motion stage can further comprise: a tilt-axis slanted linear bearing. The TnT motion stage can further comprise: a tilt-axis adjustment notch (or a tilt-axis complementary adjustment engagement component). When the tilt-axis adjustment pawl is engaged with the tilt-axis adjustment notch, a movement of the x-y motion stage along the one axis (e.g., x-axis) results in a movement of the TnT motion stage along the tilt-axis slanted linear bearing. This can result in a change in the tilt of the TnT motion stage.

Disclosed herein include embodiments of a motion platform. In some embodiments, a motion platform can comprise: a base. The motion platform can comprise: an x-y motion stage on the base. The motion platform can comprise: a tilt-axis adjustment pawl (or a tilt-axis adjustment engagement component) on the base. The motion platform can comprise: a tilt motion stage. The tilt motion stage can be on the x-y motion stage. The tilt motion stage can comprise: a tilt-axis slanted linear bearing. The tilt motion stage can comprise: a tilt-axis adjustment notch (or a tilt-axis complementary adjustment engagement component). When the tilt-axis adjustment pawl is engaged with the tilt-axis adjustment notch, a movement of the x-y motion stage along the one axis results in a movement of the tilt motion stage along the tilt-axis slanted linear bearing. This can result in a change in the tilt of the tilt motion stage. In some embodiments, the tilt motion stage can comprise: a sample carrier.

The motion platform can further comprise: a tip-axis adjustment pawl on the base. The tilt motion stage can be a tip and tilt (TnT) motion stage. The TnT motion stage can further comprise: one or more (e.g., 2, or 3, 4, 5, or more) tip-axis goniometers with different (or the same) slopes relative to one axis of the x-axis and the y-axis. The TnT motion stage can further comprise: a tip-axis adjustment notch (or a tip-axis complementary adjustment engagement component). In some embodiments, when the tip-axis adjustment pawl is engaged with the tip-axis adjustment notch, a movement of the x-y motion stage along the one axis results in a movement of the TnT motion stage along the one or more tip-axis goniometers. This can result in a change in the tip of the TnT motion stage.

Referring to FIG. 1, a motion platform can comprise an x-axis motor on (e.g., attached to, such as securely attached to) the base. The motion platform can comprise a y-axis motor on (e.g., attached to, such as securely attached to) the base. The x-axis motor can move the x-y motion stage along the x-axis. The y-axis motor can move the x-y motion stage along the y-axis. The x-axis motor and the y-axis motor can be used to change (or adjust) the tip and/or tilt of the TnT motion stage. The motion platform can comprise no additional motor other than the x-axis motor and the y-axis motor for changing (or adjusting) the tip and/or tilt of the TnT motion stage. In some embodiments, the x-axis motor is a servomotor. The y-axis motor can be a servomotor. In some embodiments, the TnT motion stage comprises no motor.

In some embodiments, the TnT motion stage is in contact with the x-y motion stage via the two tip-axis goniometers and the tilt-axis slanted linear bearing. In some embodiments, the TnT motion stage is in contact with the x-y motion stage via (e.g., via only) via the two tip-axis goniometers and the tilt-axis slanted linear bearing.

In some embodiments, the TnT motion stage comprises no motor. In some embodiments, the TnT motion stage is in contact with the x-y motion stage via the one or more first-axis goniometers and the second-axis bearing. In some embodiments, the TnT motion stage is in contact with the x-y motion stage via (e.g., only via) the one or more first-axis goniometers and the second-axis bearing.

In some embodiments, the tip-axis adjustment pawl and the tilt-axis adjustment pawl point in the opposite directions. In some embodiments, the tip-axis adjustment notch and the tilt-axis adjustment notch point in the opposite directions. In some embodiments, the tip-axis adjustment pawl and the tilt-axis adjustment pawl are elevated from the base. In some embodiments, tip-axis adjustment pawl and the tilt-axis adjustment pawl are at different heights relative to the base. The tilt-axis adjustment notch and the tip-axis adjustment notch can be at different heights relative to the base. In some embodiments, the tip-axis adjustment pawl and the tilt-axis adjustment pawl are at an identical height relative to the base. The tilt-axis adjustment notch and the tip-axis adjustment notch can be at an identical height relative to the base. In some embodiments, the tip-axis adjustment engagement component and the tilt-axis adjustment engagement component can point in the opposite directions. In some embodiments, the tip-axis complementary adjustment engagement component and the tilt-axis complementary adjustment engagement component can point in the opposite directions. In some embodiments, the tip-axis adjustment engagement component and the tilt-axis complementary adjustment engagement component can be elevated from the base. In some embodiments, the tip-axis adjustment engagement component and the tilt-axis adjustment engagement component can be at different heights relative to the base. The tilt-axis complementary adjustment engagement component and the tip-axis complementary adjustment engagement component can be at different heights relative to the base. In some embodiments, the tip-axis adjustment engagement component and the tilt-axis adjustment engagement component are at an identical height relative to the base. The tilt-axis complementary adjustment engagement component and the tip-axis complementary adjustment engagement component can be at an identical height relative to the base.

A height of a component of the motion platform, of the x-y motion stage, or of the TnT motion stage (e.g., the tip-axis adjustment engagement component, tip-axis complementary adjustment engagement component, the tilt-axis adjustment engagement component, or tilt-axis complementary adjustment engagement component) relative to the x-y motion stage can be, be about, be at least, be at least about, be at most, or be at most about, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 3 cm, 4 cm, 5 cm, 6 cm, 7 cm, 8 cm, 9 cm, 10 cm, or a number or a range between any two of these values.

In some embodiments, the two tip-axis goniometers have different slopes relative to the x-axis (or the y-axis). In some embodiments, the angle of the slope of one of the two tip-axis goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values. In some embodiments, the angle of the slope of the other of the two tip-axis goniometers can be, be about, be at least, be at least about, be at most, or be at most about, −1°, −1.1°, −1.2°, −1.3°, −1.4°, −1.5°, −1.6°, −1.7°, −1.8°, −1.9°, −2°, −2.1°, −2.2°, −2.3°, −2.4°, −2.5°, −2.6°, −2.7°, −2.8°, −2.9°, −3.0°, −3.1°, −3.2°, −3.3°, −3.4°, −3.5°, −3.6°, −3.7°, −3.8°, −3.9°, −4°, −4.1°, −4.2°, −4.3°, −4.4°, −4.5°, −4.6°, −4.7°, −4.8°, −4.9°, −5°, −5.1°, −5.2°, −5.3°, −5.4°, −5.5°, −5.6°, −5.7°, −5.8°, −5.9°, −6°, −6.1°, −6.2°, −6.3°, −6.4°, −6.5°, −6.6°, −6.7°, −6.8°, −6.9°, −7°, −7.1°, −7.2°, −7.3°, −7.4°, −7.5°, −7.6°, −7.7°, −7.8°, −7.9°, −8°, −8.1°, −8.2°, −8.3°, −8.4°, −8.5°, −8.6°, −8.7°, −8.8°, −8.9°, −9°, −9.1.°, −9.2°, −9.3°, −9.4°, −9.5°, −9.6°, −9.7°, −9.8°, −9.9°, −10°, or a number or a range between any two of these values. In some embodiments, the slopes of the two tip-axis goniometers have different absolute angles. In some embodiments, the slopes of the two tip-axis goniometers have an identical absolute angle. In some embodiments, the absolute angle of the slope of one or each of the two tip-axis goniometers is about 3.6° (see FIG. 2 for an illustration). In some embodiments, the absolute angle of the slope of one or each of the two tip-axis goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, one or each of the two tip-axis goniometers is at or adjacent to a side surface (e.g., a vertical surface relative to the platform or the x-y motion stage) of the TnT motion platform. The two tip-axis goniometers can be at or adjacent to the same side surface or different side surfaces of the TnT motion platform. In some embodiments, one or each of the two tip-axis goniometers comprises a journal and a slanted pin. The material of the journal can comprise bronze. The material of the journal can comprise bronze, aluminum, zinc, copper, titanium, tin, beryllium, bismuth, chromium, cobalt, gallium, indium, iron, manganese, nickel, rhodium, or a combination thereof. The material of the pin can comprise a steel, such as a stainless steel. A steel can be, for example, cold rolled steel, stainless steel and steel surface-treated steel. For example, a steel can be crucible steel, carbon steel, spring steel, alloy steel, maraging steel, stainless steel, high-speed steel, weathering steel, tool steel, or a combination thereof. In some embodiments, one or each of the two tip-axis goniometers comprises a magnet. The magnet can retain contact between the journal and the slanted pin.

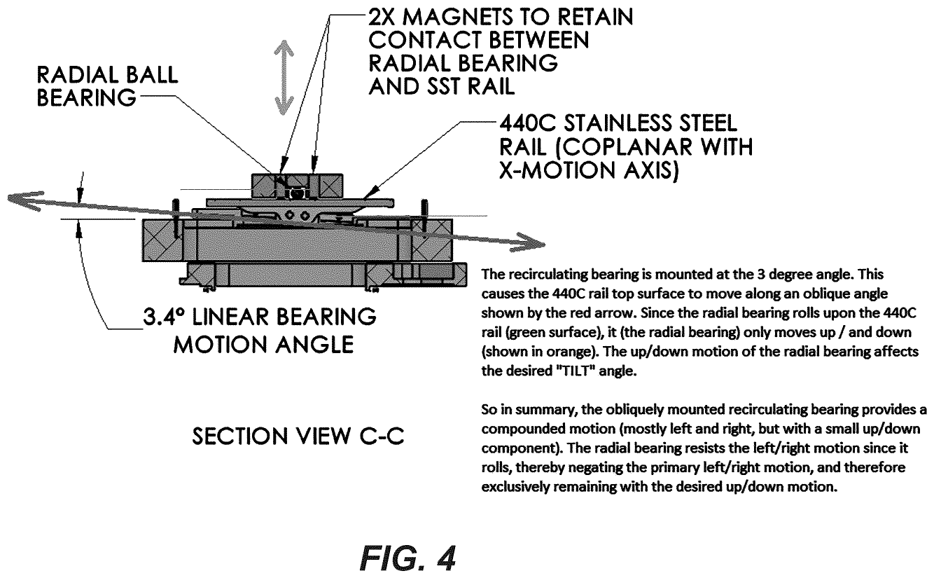

In some embodiments, the tilt-axis slanted linear bearing comprises a linear bearing carriage and a slanted linear bearing rail. In some embodiments, the linear bearing motion angle of the tilt-axis slanted linear bearing is about 3.4° (see FIGS. 3-4 for an illustration). In some embodiments, the linear bearing motion angle of the tilt-axis slanted linear bearing is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values. In some embodiments, the linear bearing motion angle of the tilt-axis slanted linear bearing is, is about, is at least, is at least about, is at most, or is at most about, −1°, −1.1°, −1.2°, −1.3°, −1.4°, −1.5°, −1.6°, −1.7°, −1.8°, −1.9°, −2°, −2.1°, −2.2°, −2.3°, −2.4°, −2.5°, −2.6°, −2.7°, −2.8°, −2.9°, −3.0°, −3.1°, −3.2°, −3.3°, −3.4°, −3.5°, −3.6°, −3.7°, −3.8°, −3.9°, −4°, −4.1°, −4.2°, −4.3°, −4.4°, −4.5°, −4.6°, −4.7°, −4.8°, −4.9°, −5°, −5.1°, −5.2°, −5.3°, −5.4°, −5.5°, −5.6°, −5.7°, −5.8°, −5.9°, −6°, −6.1°, −6.2°, −6.3°, −6.4°, −6.5°, −6.6°, −6.7°, −6.8°, −6.9°, −7°, −7.1°, −7.2°, −7.3°, −7.4°, −7.5°, −7.6°, −7.7°, −7.8°, −7.9°, −8°, −8.1°, −8.2°, −8.3°, −8.4°, −8.5°, −8.6°, −8.7°, −8.8°, −8.9°, −9°, −9.1.°, −9.2°, −9.3°, −9.4°, −9.5°, −9.6°, −9.7°, −9.8°, −9.9°, −10°, or a number or a range between any two of these values. In some embodiments, the absolute value of the linear bearing motion angle of the tilt-axis slanted linear bearing is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, the tilt-axis slanted linear bearing is at or adjacent a (or a second) side surface (e.g., a vertical surface relative to the platform or the x-y motion stage) of the TnT motion platform. In some embodiments, the TnT motion stage comprises a radial bearing in contact with a radial bearing rail. A material of the radial bearing can comprise a steel, such as a stainless steel. A material of the radial bearing rail can comprise a steel, such as a stainless steel. A steel can be cold rolled steel, stainless steel and steel surface-treated steel. A steel can comprise a steel can be crucible steel, carbon steel, spring steel, alloy steel, maraging steel, stainless steel, high-speed steel, weathering steel, tool steel, or a combination thereof. The radial bearing rail can be co-planar with the x-axis. In some embodiments, the TnT motion stage comprises at least one magnet (e.g., 2 magnets) which retains contact between the radial bearing and the radial bearing rail. In some embodiments, the radial bearing is at or adjacent to a side surface (or the second side surface). In some embodiments, the TnT motion stage comprises 8 side surfaces. In some embodiments, the TnT motion stage comprises 3, 4, 5, 6, 7, 8, 9, 10, 11, 12, 13, 14, 15, or a number or a range between any two of these values, side surfaces.

In some embodiments, when the tip-axis adjustment pawl is engaged with the tip-axis adjustment notch, the tilt-axis adjustment pawl is not engaged with the tilt-axis adjustment notch. When the tilt-axis adjustment pawl is engaged with the tilt-axis adjustment notch, the tip-axis adjustment pawl may be not engaged with the tip-axis adjustment notch. In some embodiments, when the tip-axis adjustment pawl is engaged with the tip-axis adjustment notch, a movement of the x-y motion stage along the x-axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the x-axis results in a movement of the TnT motion stage along the tilt-axis slanted linear bearing. This can result in a change in the tilt of the TnT motion stage. In some embodiments, when the tip-axis adjustment engagement component is engaged with the tip-axis complementary adjustment engagement component, the tilt-axis adjustment engagement component is not engaged with the tilt-axis complementary adjustment engagement component. When the tilt-axis adjustment engagement component is engaged with the tilt-axis complementary adjustment engagement component, the tip-axis adjustment engagement component may not be engaged with the tip-axis complementary adjustment engagement component. In some embodiments, when the tip-axis adjustment engagement component is engaged with the tip-axis complementary adjustment engagement component, a movement of the x-y motion stage along the one axis (e.g., x-axis) results in a movement of the TnT motion stage along the one or more (e.g., 2) tip-axis goniometers. This can result in a change in the tip of the TnT motion stage. In some embodiments, when the tilt-axis adjustment engagement component is engaged with the tilt-axis complementary adjustment engagement component, a movement of the x-y motion stage along the one axis (e.g., x-axis) results in a movement of the TnT motion stage along the tilt-axis bearing (e.g., tilt-axis slanted linear bearing). This can result in a change in the tilt of the TnT motion stage.

In some embodiments, when the TnT motion stage is moved along the y-axis to the edge of its travel in one direction of the y-axis, the tip-axis adjustment pawl engages with the tip-axis adjustment notch. When the TnT motion stage is moved along the y-axis to the edge of its travel in the other direction of the y-axis, the tilt-axis adjustment pawl can engage with the tilt-axis adjustment notch. In some embodiments, when the TnT motion stage is moved along one axis (e.g., the y-axis) to the edge of its travel in one direction of the axis, the tip-axis adjustment engagement component engages with the tip-axis complementary adjustment engagement component. When the TnT motion stage is moved along the axis (e.g., the y-axis) to the edge of its travel in the other direction of the axis, the tilt-axis adjustment engagement component can engage with the tilt-axis complementary adjustment engagement component.

In some embodiments, a motion platform can comprise: a base. The motion platform can comprise: an x-y motion stage. The x-y motion stage can be on (e.g., attached to, such as securely attached to) the base. The motion platform can comprise: a first-axis (e.g., a tip-axis or a tilt-axis) adjustment engagement component (e.g., a pawl or a notch) on (e.g., attached to, such as securely attached to) the base. The motion platform can comprise: a second motion stage (which can be a tip motion stage, a tilt motion stage, or a tip and tilt (TnT) motion stage). The second motion stage can be on the x-y motion stage. The motion platform can comprise: a first-axis goniometer or bearing. The motion platform can comprise: a first-axis complementary adjustment engagement component (e.g., a notch or a pawl), The first-axis adjustment engagement component and the first-axis complementary adjustment engagement component can engage (or be in engagement, such as secure engagement) with each other. For example, the first-axis adjustment engagement component and the first-axis complementary engagement component can be a pawl and a notch and can be in engagement (e.g., secure engagement) with each other. When the first-axis adjustment component is engaged with the first-axis complementary adjustment engagement component, a movement of the x-y motion stage along one axis (e.g., x-axis) of the x-axis and the y-axis can result. in a movement of the second motion stage along the first-axis goniometer or bearing. This can result in a change in the first-axis (e.g., tip-axis) of the second motion stage. In some embodiments, the motion platform can comprise: a sample carrier.

In some embodiments, the motion platform can further comprise a second-axis (e.g., a tilt-axis or a tip-axis) adjustment engagement component (e.g., a pawl or a notch). The second motion stage can further comprise: a second-axis goniometer or bearing. The second motion stage can further comprise: a second-axis complementary adjustment engagement component (e.g., a notch or a pawl). The second-axis adjustment engagement component and the second-axis complementary adjustment engagement component can engage (or be in engagement, such as secure engagement) with each other. For example, the second-axis adjustment engagement component and the second-axis complementary engagement component can be a pawl and a notch and can be in engagement (e.g., secure engagement) with each other. When the second-axis adjustment engagement component is engaged with the second-axis complementary adjustment engagement component, a movement of the x-y motion stage along the one axis can result in a movement of the second motion stage along the second-axis goniometer or bearing. This can result in a change in the second-axis (e.g., tilt-axis) of the second motion stage.

In some embodiments, the first-axis is the tip-axis. The second-axis can be the tilt-axis. In some embodiments, the first-axis is the tilt-axis. The second-axis can be the tip-axis. In some embodiments, the first-axis goniometer can comprise one or more (e.g., 2, or 3, 4, 5, or more) first-axis goniometers. In some embodiments, the first-axis bearing comprises a first-axis slanted linear bearing.

The motion platform can comprise an x-axis motor on (e.g., attached to, such as securely attached to) the base. The x-axis motor can move the x-y motion stage along the x-axis. The motion platform can comprise a y-axis motor on (e.g., attached to, such as securely attached to) the base. The y-axis motor can move the x-y motion stage along the y-axis. The motion platform can comprise no additional motor other than the x-axis motor and the y-axis motor for changing (or adjusting) the tip and/or tilt of the second motion stage. In some embodiments, the x-axis motor is a servomotor. The y-axis motor can be a servomotor.

In some embodiments, the second motion stage comprises no motor. In some embodiments, the second motion stage is in contact with the x-y motion stage via the one or more goniometers (e.g., tip-axis goniometers) and the bearing (e.g., tilt-axis slanted linear bearing). In some embodiments, the TnT motion stage is in contact with the x-y motion stage only via the one or more goniometers (e.g., tip-axis goniometers) and the bearing (e.g., tilt-axis slanted linear bearing).

In some embodiments, the first-axis adjustment engagement component and the second-axis adjustment engagement component can point in the opposite directions. In some embodiments, the first-axis complementary adjustment engagement component and the second-axis complementary adjustment engagement component can point in the opposite directions. In some embodiments, the first-axis adjustment engagement component and the second-axis complementary adjustment engagement component can be elevated from the base. In some embodiments, the first-axis adjustment engagement component and the second-axis adjustment engagement component can be at different heights relative to the base. The first-axis complementary adjustment engagement component and the second-axis complementary adjustment engagement component can be at different heights relative to the base. In some embodiments, the first-axis adjustment engagement component and the second-axis adjustment engagement component are at an identical height relative to the base. The first-axis complementary adjustment engagement component and the second-axis complementary adjustment engagement component can be at an identical height relative to the base.

In some embodiments, two goniometers have different slopes relative to the x-axis (or the y-axis). In some embodiments, the angle of the slope of one of two goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values. In some embodiments, the angle of the slope of the other of the two goniometers can be, be about, be at least, be at least about, be at most, or be at most about, −1°, −1.1°, −1.2°, −1.3°, −1.4°, −1.5°, −1.6°, −1.7°, −1.8°, −1.9°, −2°, −2.1°, −2.2°, −2.3°, −2.4°, −2.5°, −2.6°, −2.7°, −2.8°, −2.9°, −3.0°, −3.1°, −3.2°, −3.3°, −3.4°, −3.5°, −3.6°, −3.7°, −3.8°, −3.9°, −4°, −4.1°, −4.2°, −4.3°, −4.4°, −4.5°, −4.6°, −4.7°, −4.8°, −4.9°, −5°, −5.1°, −5.2°, −5.3°, −5.4°, −5.5°, −5.6°, −5.7°, −5.8°, −5.9°, −6°, −6.1°, −6.2°, −6.3°, −6.4°, −6.5°, −6.6°, −6.7°, −6.8°, −6.9°, −7°, −7.1°, −7.2°, −7.3°, −7.4°, −7.5°, −7.6°, −7.7°, −7.8°, −7.9°, −8°, −8.1°, −8.2°, −8.3°, −8.4°, −8.5°, −8.6°, −8.7°, −8.8°, −8.9°, −9°, −9.1.°, −9.2°, −9.3°, −9.4°, −9.5°, −9.6°, −9.7°, −9.8°, −9.9°, −10°, or a number or a range between any two of these values. In some embodiments, the slopes of the two goniometers have different absolute angles. In some embodiments, the slopes of the goniometers have an identical absolute angle. In some embodiments, the absolute angle of the slope of one or each of the two goniometers is about 3.6° (see FIG. 2 for an illustration). In some embodiments, the absolute angle of the slope of one or each of the two goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, one or each of the two goniometers is at or adjacent to a side surface (e.g., a vertical surface relative to the platform or the x-y motion stage) of the TnT motion platform. The two goniometers can be at or adjacent to the same side surface or different side surfaces of the second motion platform. In some embodiments, one or each of the two goniometers comprises a journal and a slanted pin. A material of a component herein (e.g., a journal, a pin, a carriage, or a rail) can comprise bronze, aluminum, zinc, copper, titanium, tin, beryllium, bismuth, chromium, cobalt, gallium, indium, iron, manganese, nickel, rhodium, or a combination thereof. A material of the component can comprise a steel, such as cold rolled steel, stainless steel and steel surface-treated steel. A steel can comprise a steel can be crucible steel, carbon steel, spring steel, alloy steel, maraging steel, stainless steel, high-speed steel, weathering steel, tool steel, or a combination thereof. In some embodiments, one or each of the two goniometers comprises a magnet. The magnet can retain contact between the journal and the slanted pin.

In some embodiments, the bearing can be linear bearing (e.g., a slanted linear bearing). The bearing can comprise a carriage and a rail (e.g., a slanted rail). In some embodiments, the bearing motion angle is about 3.4° (see FIGS. 3-4 for an illustration). In some embodiments, the bearing motion angle is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values. In some embodiments, the bearing motion angle is, is about, is at least, is at least about, is at most, or is at most about, −1°, −1.1°, −1.2°, −1.3°, −1.4°, −1.5°, −1.6°, −1.7°, −1.8°, −1.9°, −2°, −2.1°, −2.2°, −2.3°, −2.4°, −2.5°, −2.6°, −2.7°, −2.8°, −2.9°, −3.0°, −3.1°, −3.2°, −3.3°, −3.4°, −3.5°, −3.6°, −3.7°, −3.8°, −3.9°, −4°, −4.1°, −4.2°, −4.3°, −4.4°, −4.5°, −4.6°, −4.7°, −4.8°, −4.9°, −5°, −5.1°, −5.2°, −5.3°, −5.4°, −5.5°, −5.6°, −5.7°, −5.8°, −5.9°, −6°, −6.1°, −6.2°, −6.3°, −6.4°, −6.5°, −6.6°, −6.7°, −6.8°, −6.9°, −7°, −7.1°, −7.2°, −7.3°, −7.4°, −7.5°, −7.6°, −7.7°, −7.8°, −7.9°, −8°, −8.1°, −8.2°, −8.3°, −8.4°, −8.5°, −8.6°, −8.7°, −8.8°, −8.9°, −9°, −9.1.°, −9.2°, −9.3°, −9.4°, −9.5°, −9.6°, −9.7°, −9.8°, −9.9°, −10°, or a number or a range between any two of these values. In some embodiments, the absolute value of the bearing motion angle is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, the bearing is at or adjacent a (or a second) side surface (e.g., a vertical surface relative to the platform or the x-y motion stage) of the second motion platform. In some embodiments, the second motion stage (or the bearing) can comprise a bearing in contact with a bearing rail. In some embodiments, the second motion stage (or the bearing) can comprise a radial bearing in contact with a radial bearing rail. The radial bearing rail can be co-planar with the x-axis. In some embodiments, the second motion stage comprises at least one magnet (e.g., 2, or 3, 4, 5, or more, magnets) which retains contact between the radial bearing and the radial bearing rail. In some embodiments, the radial bearing is at or adjacent to a side surface (or the second side surface that is different from the surface the goniometer is adjacent to). In some embodiments, the second motion stage comprises 8 side surfaces. In some embodiments, the second motion stage comprises 3, 4, 5, 6, 7, 8, 9, 10, 11, 12, 13, 14, 15, or a number or a range between any two of these values, side surfaces.

In some embodiments, when the first-axis adjustment engagement component is engaged with the first-axis complementary adjustment engagement component, the second-axis adjustment engagement component is not engaged with the second-axis complementary adjustment engagement component. When the second-axis adjustment engagement component is engaged with the second-axis complementary adjustment engagement component, the first-axis adjustment engagement component may not be engaged with the first-axis complementary adjustment engagement component. In some embodiments, when the first-axis adjustment engagement component is engaged with the first-axis complementary adjustment engagement component, a movement of the x-y motion stage along the one axis (e.g., x-axis) results in a movement of the second motion stage along the one or more (e.g., 2) tip-axis goniometers. This can result in a change in the first axis (e.g., the tip) of the TnT motion stage. In some embodiments, when the second-axis adjustment engagement component is engaged with the second-axis complementary adjustment engagement component, a movement of the x-y motion stage along the one axis (e.g., x-axis) results in a movement of the second motion stage along the second-axis bearing (e.g., second-axis slanted linear bearing). This can result in a change in the second-axis (e.g., the tilt) of the TnT motion stage.

In some embodiments, when the second motion stage is moved along one axis (e.g., the y-axis) to the edge of its travel in one direction of the axis, the first-axis adjustment engagement component engages with the first-axis complementary adjustment engagement component. When the second motion stage is moved along the axis (e.g., the y-axis) to the edge of its travel in the other direction of the axis, the second-axis adjustment engagement component can engage with the second-axis complementary adjustment engagement component.

In some embodiments, the ringdown time (e.g., of the motion platform or the x-y motion stage disclosed herein) is, is about, is at least, is at least about, is at most, or is at most about, 5 milliseconds (ms), 6 ms, 7 ms, 8 ms, 9 ms, 10 ms, 11 ms, 12 ms, 13 ms, 14 ms, 15 ms, 16 ms, 17 ms, 18 ms, 19 ms, 20 ms, 21 ms, 22 ms, 23 ms, 24 m, 25 ms, 26 ms, 27 ms, 28 ms, 29 ms, 30 ms, 35, ms, 40 ms, 45 ms, 50 ms, 55 ms, 60 ms, 65 ms, 70 ms, 75 ms, 80 ms, 85 ms, 90 ms, or a number or a range between any two of these values. In some embodiments, the motion time (e.g., of the motion platform or the x-y motion stage disclosed herein) is, is about, is at least, is at least about, is at most, or is at most about, 70 milliseconds (ms), 75 ms, 80 ms, 85 ms, 90 ms, 95 ms, 100 ms, 105 ms, 110 ms, 115 ms, 120 ms, 125 ms, 130 ms, 135 ms, 140 ms, 145 ms, 150 ms, 155 ms, 160 ms, 165 ms, 170 ms, 175 ms, 180 ms, or a number or a range between any two of these values. A design of the present disclosure can have a throughput improvement (relative to the throughput of a prior design) of, of about, of at least, of at least about, of at most, or of at most about, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, or a number or a range between any two of these values. In some embodiments, the presently disclosed mechanism's net weight to achieve the TnT functionality can be, be about, be at least, be at least about, be at most, or be at most about, 100 grams (g), 110 g, 120 g, 130 g, 140 g, 150 g, 160 g, 170 g, 180 g, 190 g, 200 g, 225 g, 250 g, 275 g, 300 g, 325 g, 350 g, 375 g, 400 g, 425 g, 450 g, 475 g, 500 g, or a number or a range between any two of these values.

### Exemplary Instruments

Disclosed herein include embodiments of an instrument. In some embodiments, the instrument can comprise: a sensor (e.g., below or above the motion platform). The instrument can comprise: optics (e.g., below or above the motion platform). The instrument can comprise: a motion platform disclosed herein. The instrument can comprise a fluorescent imaging system, such as an optical genome mapping (OGM) system. In some embodiments, the motion platform is suspended within the imaging system.

### Exemplary Motion Platform Uses

Disclosed herein include embodiments of a method of positioning a sample (e.g., adjusting the tip-axis and the tilt axis of the sample or TnT motion stage). In some embodiments, a method of positioning a sample can comprise: providing a sample. The sample can be in a sample chip or cartridge. The method can comprise: placing the sample, or the sample chip or cartridge, on (or onto or into) a sample carrier of a TnT motion stage of a motion platform of the present disclosure. The method can comprise: engaging the tip-axis adjustment paw (or tip adjustment engagement component) with the tip-axis adjustment notch (or complementary tip adjustment engagement component). The method can comprise: moving the x-y motion stage along one axis (e.g., the y-axis) of the x-axis and the y-axis. This can result in changing the tip of the TnT motion stage. The method can include: engaging the tilt-axis adjustment paw (or tilt adjustment engagement component) with the tilt-axis adjustment notch (or complementary tilt adjustment engagement component). The method can include: moving the x-y motion stage along the one axis (e.g., the y-axis) of the x-axis and the y-axis. This can result in changing the tilt of the TnT motion stage. Prior to engaging the tilt-axis adjustment paw with the tilt-axis adjustment notch, the method can include: disengaging the tip-axis adjustment paw with the tip-axis adjustment notch.

In some embodiments, engaging the tip-axis adjustment paw with the tip-axis adjustment notch and moving the x-y motion stage along the one axis of the x-axis and the y-axis occurs before engaging the tilt-axis adjustment paw with the tilt-axis adjustment notch and moving the x-y motion stage along the one axis of the x-axis and the y-axis, thereby changing the tilt of the TnT motion stage. Prior to engaging the tilt-axis adjustment paw with the tilt-axis adjustment notch, the method can include: disengaging the tip-axis adjustment paw with the tip-axis adjustment notch. In some embodiments, engaging the tip-axis adjustment paw with the tip-axis adjustment notch and moving the x-y motion stage along the one axis of the x-axis and the y-axis occurs after engaging the tilt-axis adjustment paw with the tilt-axis adjustment notch and moving the x-y motion stage along the one axis of the x-axis and the y-axis, thereby changing the tilt of the TnT motion stage. Prior to engaging the tip-axis adjustment paw with the tip-axis adjustment notch, the method can include: disengaging the tilt-axis adjustment paw with the tilt-axis adjustment notch. In some embodiments, the method comprises: changing the x-y position of the motion stage. Changing the x-y position of the motion stage can occur before or after changing the tip of the TnT motion stage and/or the tilt of the TnT motion stage.

In some embodiments, a method of positioning a sample can comprise: providing a sample. The method can comprise: placing the sample, or a sample chip or cartridge comprising the sample, on (or onto or into) a sample carrier of a TnT motion stage of a motion platform of the present disclosure. The method can include: determining a tip-tilt adjustment needed for the sample. The method can include: engaging a tip-axis adjustment paw (or tip adjustment engagement component) on the base of the motion platform with a tip-axis adjustment notch (or complementary tip adjustment engagement component) of the TnT motion stage. The method can include: moving an x-y motion stage of the motion platform along one axis of the x-axis and the y-axis, thereby changing the tip of the TnT motion stage, based on the tip-tilt adjustment needed. The method can include engaging a tilt-axis adjustment paw (or tilt adjustment engagement component) on the base of the motion platform with a tilt-axis adjustment notch (or complementary tilt adjustment engagement component) of the TnT motion stage. The method can include: moving the x-y motion stage along the one axis of the x-axis and the y-axis, thereby changing the tilt of the TnT motion stage, based on the tip-tilt adjustment needed.

In some embodiments, a method of positioning a sample can comprise: providing a sample. The method can comprise: placing the sample, or a sample chip or cartridge comprising the sample, on (or onto or into) a sample carrier of a TnT motion stage of a motion platform of the present disclosure. The method can comprise a iterative (or stepwise) process. The iterative process can comprise: determining a tip-tilt adjustment needed for the sample. The iterative process can include: engaging a tip-axis adjustment paw (or tip adjustment engagement component) on the base of the motion platform with a tip-axis adjustment notch (or complementary tip adjustment engagement component) of the TnT motion stage. The iterative process can include: moving an x-y motion stage of the motion platform along one axis of the x-axis and the y-axis, thereby changing the tip of the TnT motion stage, based on the tip-tilt adjustment needed. The iterative process can include: engaging a tilt-axis adjustment paw (or tilt adjustment engagement component) on the base of the motion platform with a tilt-axis adjustment notch (or complementary tilt adjustment engagement component) of the TnT motion stage. The iterative process can include: moving the x-y motion stage along the one axis of the x-axis and the y-axis, thereby changing the tilt of the TnT motion stage, based on the tip-tilt adjustment needed.

In some embodiments, the iterative process comprises: determining a chip gradient. The iterative process can comprise: engaging a tip-axis adjustment paw on the base of the motion platform with a tip-axis adjustment notch of the TnT motion stage. The iterative process can comprise: moving an x-y motion stage of the motion platform along one axis of the x-axis and the y-axis, thereby changing the tip of the TnT motion stage, based on the chip gradient. The iterative process can comprise: engaging a tilt-axis adjustment paw on the base of the motion platform with a tilt-axis adjustment notch of the TnT motion stage. The iterative process can comprise: moving the x-y motion stage along the one axis of the x-axis and the y-axis, thereby changing the tilt of the TnT motion stage, based on the chip gradient adjustment needed. The iterative process can comprise: determining a FOV gradient. The iterative process can comprise: engaging a tip-axis adjustment paw on the base of the motion platform with a tip-axis adjustment notch of the TnT motion stage. The iterative process can comprise: moving an x-y motion stage of the motion platform along one axis of the x-axis and the y-axis, thereby changing the tip of the TnT motion stage, based on the FOV gradient. The iterative process can comprise: engaging a tilt-axis adjustment paw on the base of the motion platform with a tilt-axis adjustment notch of the TnT motion stage. The iterative process can comprise: moving the x-y motion stage along the one axis of the x-axis and the y-axis, thereby changing the tilt of the TnT motion stage based on the FOV gradient adjustment needed.

In some embodiments, a method of positioning a sample comprises: (a) providing a sample. The sample can be in a sample chip or cartridge. The method can comprise: (b) placing the sample, or the sample chip or cartridge, on (or onto or into) a sample carrier of a tip and tilt (TnT) motion stage of a motion platform of the present disclosure. The TnT motion stage can be on an x-y motion stage of the motion platform. The x-y motion stage can be on a base of the motion platform. The method can comprise: (c1) determining a chip gradient of the sample. The method can comprise: (d1) performing one or more steps of the following based on the chip gradient in step (d1). The method can comprise: engaging a tip-axis adjustment paw (or tip adjustment engagement component) on the base of the motion platform with a tip-axis adjustment notch (or complementary tip adjustment engagement component) of the TnT motion stage. The method can comprise: moving an x-y motion stage of the motion platform along one axis of the x-axis and the y-axis, thereby changing the tip of the TnT motion stage. The method can comprise: engaging a tilt-axis adjustment paw (or tilt adjustment engagement component) on the base of the motion platform with a tilt-axis adjustment notch (or complementary tilt adjustment engagement component) of the TnT motion stage. The method can comprise: moving the x-y motion stage along the one axis of the x-axis and the y-axis, thereby changing the tilt of the TnT motion stage. In some embodiments, the method can comprise: (c2) determining a field of view (FOV) gradient. The method can comprise: (d2) performing one or more steps of (d1) based on the FOV gradient.

In some embodiments, a method of positioning a sample comprises: providing a sample. The sample can be in a sample chip or cartridge. The method can include: placing the sample, or the sample chip or cartridge) on (or onto or into) a sample carrier of a tip and tilt (TnT) motion stage of a motion platform of the present disclosure. The TnT motion stage can be on an x-y motion stage of the motion platform. The x-y motion stage can be on a base of the motion platform. The method can include: determining a chip gradient of the sample. The method can include: engaging a tip-axis adjustment paw (or tip adjustment engagement component) on the base of the motion platform with a tip-axis adjustment notch (or complementary tip adjustment engagement component) of the TnT motion stage and moving an x-y motion stage of the motion platform along one axis of the x-axis and the y-axis, thereby changing the tip of the TnT motion stage, based on the chip gradient. The method can include: engaging a tilt-axis adjustment paw (or tilt adjustment engagement component) on the base of the motion platform with a tilt-axis adjustment notch (or complementary tilt adjustment engagement component) of the TnT motion stage and moving the x-y motion stage along the one axis of the x-axis and the y-axis, thereby changing the tilt of the TnT motion stage, based on the chip gradient. The method can include: determining a field of view (FOV) gradient. The method can include: engaging the tip-axis adjustment paw with the tip-axis adjustment notch and moving an x-y motion stage of the motion platform along the one axis of the x-axis and the y-axis, thereby changing the tilt of the TnT motion stage, based on the FOV gradient. The method can include: engaging the tilt-axis adjustment paw with the tilt-axis adjustment notch and moving the x-y motion stage along the one axis of the x-axis and the y-axis, thereby changing the tilt of the TnT motion stage, based on the FOV gradient.

Disclosed herein include methods of imaging a sample. In some embodiments, a method of imaging a sample comprising: positioning (e.g., adjusting the tip-axis and/or tilt-axis) a sample as described herein. The sample can be on (or in) a sample carrier of a TnT motion stage of a motion platform of the present disclosure. Positioning the sample can include adjusting the tip-axis and/or tilt-axis of the TnT motion stage as described herein. The method can include: rastering, using the x-y motion stage, to different positions along the x-axis and/or the y-axis. The number of positions can be different in different embodiments, such as 2500, 5000, 7500, 10000, 25000, 50000, 75000, 100000, or a number or a range between any two of these values. The number of images captured can be different in different embodiments, such as 2500, 5000, 7500, 10000, 25000, 50000, 75000, 100000, or a number or a range between any two of these values. The method can include capturing images of the sample at the different positions. Attenuation of resonance (e.g., resonance of the motion platform or components thereof, such as the x-y motion stage and/or the TnT motion stage) can occur first prior to imaging. Attenuation of resonance can occur when the resonance is, for example, less than 20 nm, 25 nm 30 nm, 35 nm, 40 nm, 45 nm, 50 nm or more or less. In some embodiments, the tip-axis and/or tilt-axis of the sample (or the TnT motion stage) can be adjusted after a number of images of the sample are captured at different positions. The number of images captured prior to tip axis and/or tilt-axis being adjusted again can be different in different embodiments, such as 100, 250, 500, 750, 1000, 2500, 5000, 7500, 10000, 25000, 50000, 75000, 100000, or a number or a range between any two of these values. In some embodiments, the tip-axis and/or tilt-axis of the sample (or the TnT motion stage) may not need to be adjusted.

In some embodiments, the sample comprises an optical genome mapping (OGM) sample. In some embodiments, the sample comprises nucleic acids. The nucleic acids can comprise deoxyribonucleic acid (DNA). The nucleic acids can comprise the nucleic acids comprise genomic DNA. The nucleic acids can comprise fragmented genomic DNA. The nucleic acids can comprise ribonucleic acids (RNA). The nucleic acids can comprise DNA derived (e.g., reverse transcribed) from DNA or RNA. In some embodiments, the sample comprises labeled nucleic acids, optionally wherein the sample comprises fluorescently labeled nucleic acids.

### Exemplary Tip and Tilt Motion Stage Control

### 1. Introduction

In a true goniometer, an object can be rotated to an exact angular position with respect to an origin. In the novel design described herein, the geometry is more complicated as described herein such that the term “gonio” may not mean rotating an object to an exact angular position.

The gonio stage (also referred to herein as a tip and tilt (TnT) motion stage) can be used to apply a gradient to the currently loaded chip, to level the currently loaded chip with the imaging focal plane so that molecules and labels stay in focus. For the Y (tilt axis) there is a rotation about a center, so this Y axis more closely resembles a goniometer. For the X (tip axis), a sliding motion on opposing ramps imparts a slope, rather than a rotation. The stage design described herein has another major difference from previous designs which affects all aspects of the adjustment determinations and control of the stage. For the stage design, the tip tilt stage is on top of the x, y stage, while for previous designs the x, y stage is on top of the tip tilt stage.

2a. Gradient Application and Definitions

The FOV gradient in the TnT motion stage described herein is defined as the slope between the stage plane of motion and the imaging focal plane, represented by QF.

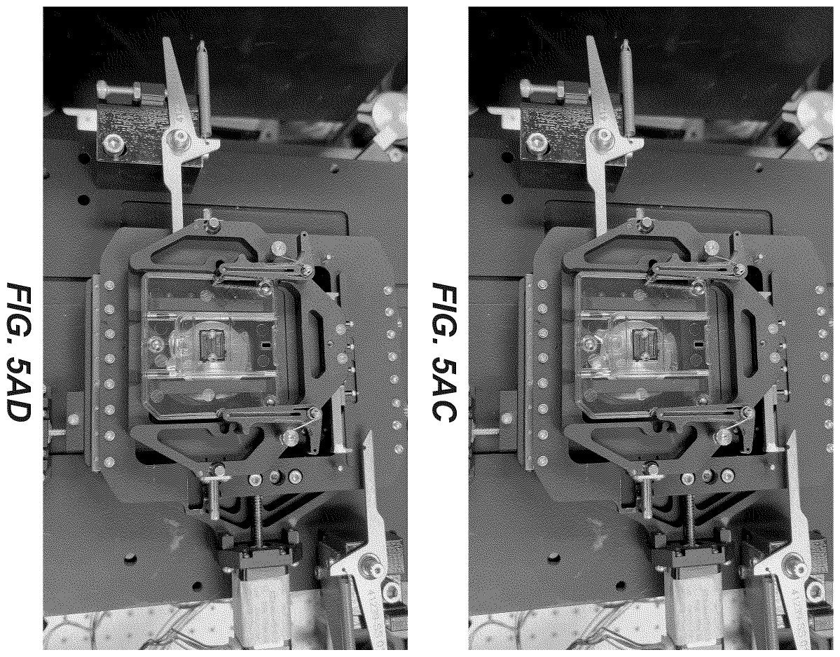

The chip (which can comprise a labeled sample, such as a OGM sample) gradient in the TnT motion stage described herein is defined as the slope between the current chip and the stage plane of motion, represented by qc. FIG. 6. Exemplary gradient planes and angles definitions.

A key difference between the stage and chip gradient is that in order to apply these, the negative of the chip gradient is applied to “level” the chip. While for the FOV gradient, the positive of the gradient is applied to move the chip to the nominal focal plane. These sign differences lead to confusion at times.

Since the FOV gradient contribution is positive and the chip gradient contribution is negative, to level the chip to the focal plane, the correction to apply will be called the leveling gradient φ_{L} and is defined the difference between the FOV gradient and chip gradient.

$${ϕ}_{L} = {ϕ}_{F} - {ϕ}_{C} \qquad Eq ( 1 )$$

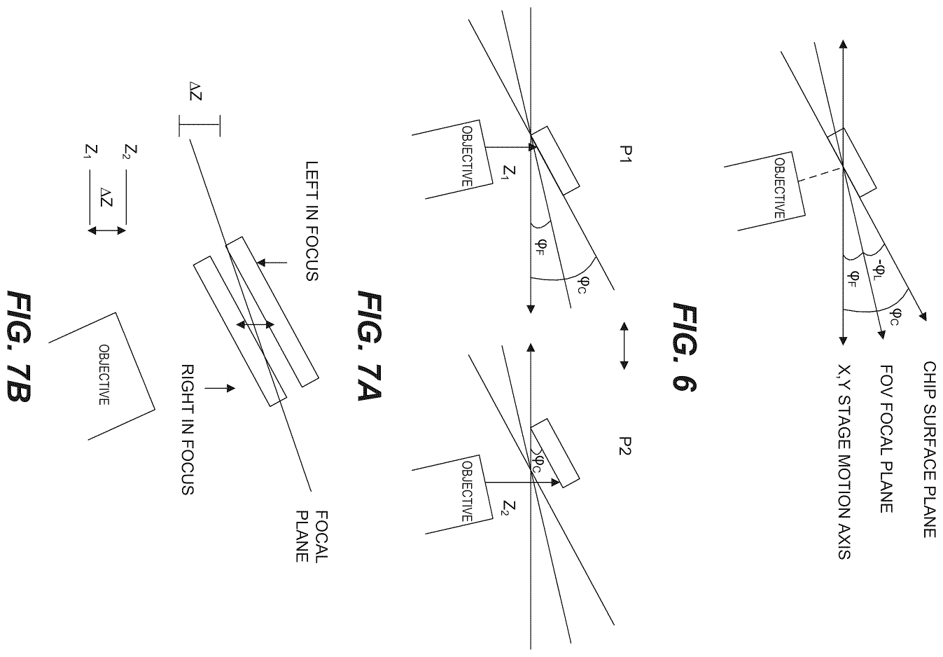

Upon starting a new chip inspection, the chip is leveled. An instrument containing the motion platform (or a component thereof, such as the TnT motion stage) can be equipped with a laser proximeter which measures the distance of the chip surface in the z axes away from the objective. This proximeter can be referred to herein as the focuser. The position of the chip in several corners can be measured using an X, Y stage shift and the focuser during chip alignment, and the chip gradient, or φ_{C} is measured. This is because the chip still moves about the stage original plane unlike previous implementations, where the stage plane shifts upon application of the stage gradient. The plane of motion of the chip never shifts in some embodiments. FIG. 7A. Exemplary leveling measurement during alignment measures chip gradient φ_{C}.

When a gradient is measured using an image stack in the Z direction, what is measured is the distance between the chip and the focal plane, which then is the leveling gradient φ_{L} (see the definition herein). FIG. 7B. Exemplary image Z stack measures the difference between the chip plane and the FOV focal plane, φ_{L}. An image stack can comprise a plurality of images, such as 2, 3, 4, 5, 10, 15, 20, 30, 40, 50, or more or fewer, images. The gradient which is applied scan time can be the leveling gradient φ_{L}. This can be visualized by rotating the chip in FIG. 6 by φ_{L} in the negative (clockwise direction), which levels it to the objective plane of focus.

Once the FOV gradient is established, then during the first phase of chip alignment, the chip is not yet leveled and the leveling gradient is not known. The tip tilt gradient can be set to the FOV gradient because the chip gradient is assumed as zero, thus using equation 1 above. This would shift the nominal “perfect” chip into focus.

After alignment levels the chip, this process can now calculate the chip gradient, as explained above. From the chip gradient, the leveling gradient can be calculated using the premeasured FOV gradient, again using equation 1. This calculated leveling gradient φ_{L} is applied to the stage.

2b. Exemplary Gradient Setup

An exemplary method of FOV gradient setup for a design disclosed herein is as the follows.

1. Clear chip and FOV gradient to zero and apply.

2. Align chip to measure chip gradient.

3. At this point the FOV gradient is set to zero. Calculate φ_{C} and φ_{L}=−φc so the negative of φC is applied.

4. Run FOV gradient image stack measurement. This calculates the difference between the current chip position and the FOV focal plane. The difference is φL. This measurement is the negative of the FOV gradient (see FIG. 6 and equation (1)). The negative of this measured gradient is saved as the pending FOV gradient and leveling gradient is recalculated and displayed as pending as well.

5. Apply and save the new FOV gradient.

6. Run the FOV gradient check again and the change in FOV gradient should be small.

### 3. Gonio Axes Geometry and Gradient Calculations

a. General/Terminology

The application of the X axis (tip) portion of the gradient can be performed by shifting the X position of a sliding assembly which rides on sloped rails in X. This also confers an X axis shift of the chip position to be image. The Y portion of the gradient can be applied via a rotation about these rails in the Y direction. The shift in Y is much smaller than the shift in X, but still non-zero for the rotation around these rails and the standoff of the chip surface above these rails. To apply a gradient, transforms such as mathematical transforms can be used to calculate or determine, for the desired X, Y gradient, an output gonio X and Y coordinate which imparts such a gradient. These transforms can be non-trivial due to the design because the X axis is not a true goniometer (a true goniometer would be purely rotational).

The gradient discussed here is a true 2D x, y gradient, which is the dimensionless “slope” in the X and Y directions on the plate. This is not the same as the angle, but for small angles these are close (small angle approximations). When trigonometric computations are performed, angle (in radians) can be used. When other computations such as vector computations are used, slope or gradient can be used. It will be clear by naming conventions when each is being referenced (e.g., θ, φ for angle, ∇ for gradient, etc.).

b. Hardware Configuration Parameters.

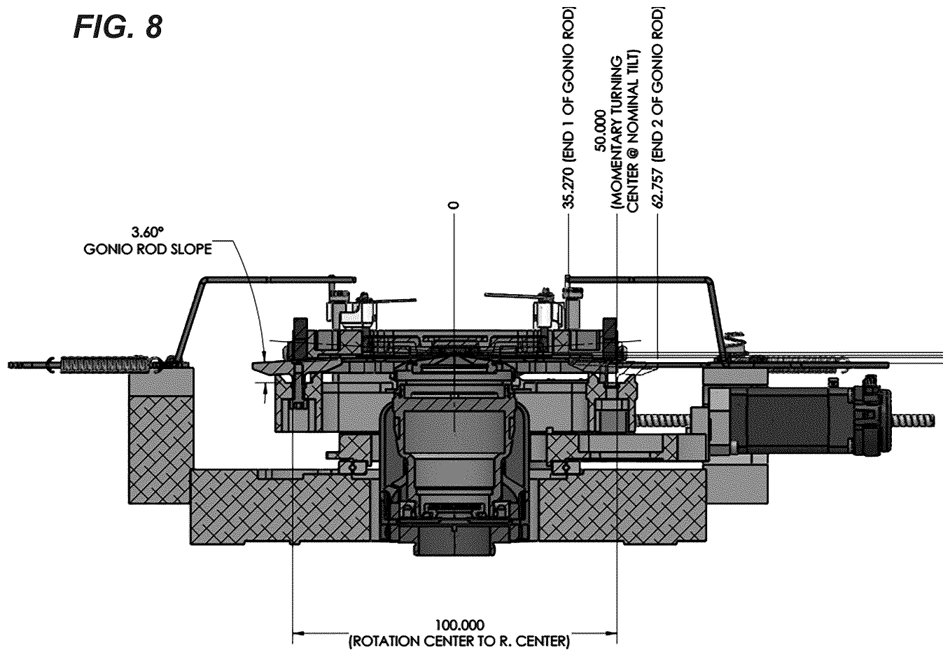

The tip tilt stage has ideal design parameters that can be used to calculate (or determine) the coordinate transformations used internally. These parameters are specified as follows (see FIG. 8).

The chip center position when mounted in the caddy holder (or sample carrier) may not be at center.

The X plate diameter is from rail slider to rail.

The Y plate diameter is 2* the radius from Y bearing contact point to the Y at the X.

The X rail ramp is the angle in degrees.

The Y rail ramp is the angle in degrees.

The standoff is the distance from the contact point of the X rail centers beneath the sliders up to the imaging surface of the chip.

The Y axis bearing position is the distance along the X axis off-center of Y rod/bearing.

The X and Y stage travel range may not be used internally in the calculations, but may need to be applied to clamp the range applied by, for example, the higher level software.

c. Vector Calculations Plate Vector Parametric Equations

Much of the calculations used to calculate the coordinate transforms can be accomplished through vector calculations using parametric equations, in some embodiments. An example parametric line equation using an n dimensioned vector P, between two end points would be as below. In calculations for the X and the Y axis, this can be a 2D vector with either X or Y as the first dimension, and Z as the second dimension.

$$\vec{P} = {\vec{P}}_{0} + ( {\vec{P}}_{1} - {\vec{P}}_{0} ) t , 0 \leq t \leq 1$$

Plate vectors can be calculated along the stage constraints described herein. These vectors are along the surface of the theoretical plate but modified by the stage constraints. There is no physical plate at this location. For the X axis, the plate bottom can be considered the center of the rails where the bearing sliders rotate about. For the Y axis, the plate can be considered the extension from the x axis where the rail on the Y axis raises the theoretical plate. Above this theoretical plate, a standoff height above this plate defines the chip plane. See the drawings for the individual axes below for a more detailed illustration.

In some embodiments, the gonio X, Y are offsets from the ideal gonio stage center position with 0 gradient.

d. Planar Coordinate Reference Positions.

A “Planar Transform” can be calculated from the three contact positions of the gonio bundle, as shown in FIG. 9, Z1, Z2, Z3. Unlike previous stage implementations, in this planar transform, the x and y axis position of these three points may not be fixed, but shift in x with the application of the x gradient, since the sliders move along the rail in real space.

### 4. Gonio X-Axis

a. Internal X, Z Coordinate System

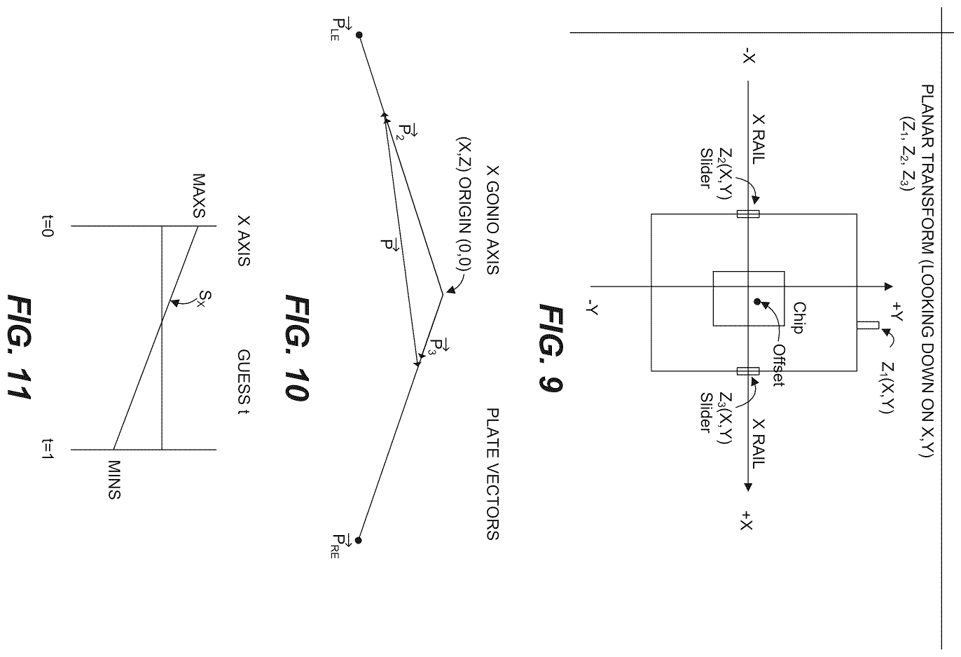

The origin of the gonio X axis coordinate system can be defined by a 0.0 point which is at the virtual apex (meeting point) of the x rails center, if the x rails continued to the middle. This arrangement can simplify the parametric equations for the X axis constraints. The end points \vec{P}_{Le} and \vec{P}_{Re} (Left End and Right End) can be the positions with the plate at a limit where the opposite end would be at the apex at the virtual origin. So the plate vectors at these end locations would be coincident with the rails at these end points. This may not be the true range, which is practically less, just the calculation limits for the equations. FIG. 10. Exemplary internal X, Z coordinate system.

With this layout, the end point vectors can be calculated from the ramp slope.

$${X}_{L} = L cos ( φ )$$

$${Y}_{L} = L sin ( φ )$$

$${\vec{P}}_{Le} = [ \begin{matrix}- {X}_{L} \\ - {Y}_{L}\end{matrix} ]$$

$${\vec{P}}_{R e} = [ \begin{matrix}{X}_{L} \\ - {Y}_{L}\end{matrix} ]$$

b. Parametric Vector Equations

With this simplified coordinate system origin, the plate vector parametric equations can be simplified as:

$${\vec{P}}_{2} = {\vec{P}}_{L e} ⋆ ( 1 - t ) \qquad 0 <= t < = 1$$

$${\vec{P}}_{3} = {\vec{P}}_{R e} ⋆ t \qquad L = X dimension plate length$$

$$\vec{P} = {\vec{P}}_{3} - {\vec{P}}_{2}$$

Here, at t=0, plate is far left, max slope. At t=1, plate is far right, min slope.

c. Calculating Parametric t

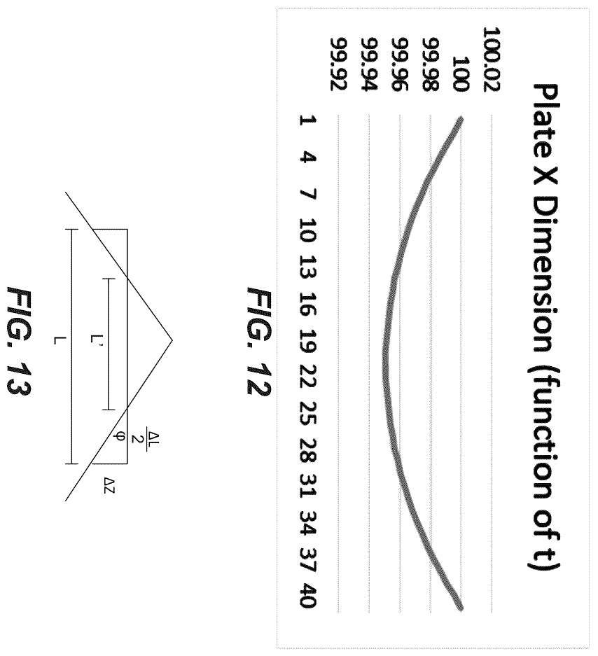

The t parameter of the parametric equations can be used to calculate the gradient applied. With the end points specified, t goes from max slope at the far left theoretical ramp position at \vec{P}_{Le}, to the min slope position \vec{P}_{Re} as t transverses from 0 to 1. This allows calculating t as a function of x gradient (slope). FIG. 11. Exemplary parametric t calculation.

d. Z Height Correction for Plate Shortening at Ramp Constraints

The constraints of the plate vector calculated may not be precise. A further constraint can be that the plate length is constant. The actual plate length as calculated by this approximation can vary and a small correction my be used for precision of the true plate position.

At the extremes of slope, the plate is full length in these constraints, but it shortens towards the middle (zero slope) (FIG. 12).

The effect of this shortening is that the actual plate can be longer, and thus the plate vector can lower in z in order to make contact with the rails. This correction can be calculated through geometry, approximately but accurate because the correction and plate angle can be very small.

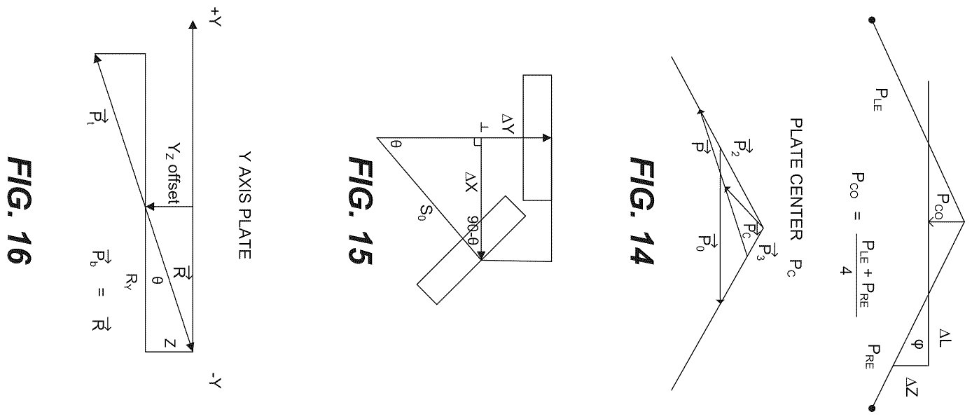

The angle φ is the angle between the plate the rail, but can be approximated as just the rail angle because the left side and right side z shift will cancel and the plate can be constrained in the X position by the stage gradient. FIG. 13. Exemplary φ approximation. In FIG. 13:

$$Δ L = ( \vert \vec{P} \vert - L ) / 2$$

$$Δ Z ≅ Δ L tan ( φ )$$

e. Stage X Offset to Apply X Component of Gradient

One goal of the calculation is to determine the amount of x stage offset \vec{P}_{stagex} needed to apply a specified gradient. The amount of shift in the X axis and resulting z position can be calculated from the plate vectors. The shift in the plate position can be calculated using the nominal center vector \vec{P}_{c0} and the new shifted plate vector center \vec{P}_{c}. The total x, z shift vector is then:

$${\vec{P}}_{c 0} = ( {\vec{P}}_{L e} + {\vec{P}}_{R e} ) / 4$$

$${\vec{P}}_{c} = ( {\vec{P}}_{2} + {\vec{P}}_{3} ) / 2$$

$$Shift vector {\vec{P}}_{stagex} = {\vec{P}}_{c} - {\vec{P}}_{c 0}$$

FIG. 14. Exemplary stage X offset to apply X component of gradient.

f. X, Z Correction for Translational and Rotational Shift.

The chip shift in imaging position can be calculated with translation shift and rotational shift terms.

The translational shift x, z component can be the same as the shift vector \vec{P}_{stagex}.

The rotational shift x, z component can be calculated by the standoff S₀ of the chip above the rotation axis. FIG. 15. Exemplary rotational shift x and z components. In FIG. 15,

$$Δ x = {S}_{0} sin ( θ )$$

$$Δ z = {S}_{0} - {S}_{0} cos ( θ )$$

$$Δ z = {S}_{0} ( 1 - cos ( θ ) )$$

$${\vec{P}}_{r o t a t ionalx} = [ \begin{matrix}Δ x \\ Δ z\end{matrix} ]$$

$$Total x , z clip shift = {\vec{P}}_{s t a g e x} + {\vec{P}}_{rotationalx}$$

### 5. Gonio Y-Axis

a. Internal Y, Z Coordinate System and Crosstalk Issues

The Y coordinate system follows the X coordinate system Z origin. This can require adding in a final offset to position intermediate Z calculations into this X axis system.

An important consequence of a design disclosed herein has the Y bearing position off center of the Y axis. This causes “crosstalk” in the axes such that when an X gradient is applied, the Y position can need to be shifted to maintain the Y gradient required. These calculations are shown below.

The amount of shift to correct the Y gradient can be calculated by using the X plate vectors, since it is a function of the current X slope, using the X position of the Y axis bearing. This correction can follow the slope of the X plane at the Y axis offset in X.

$$YZoffset = \vec{P} \cdot Y * ( \frac{YaxisX}{\vert \vec{P} \vert} )$$

b. Parametric Vector Equations

For the Y axis, the t parameter can be, for example, chosen as the Z offset from center at the Y bearing.

c. Plate Vector Calculations

The nominal Y plate radius R_{y}. As the Y is raised up or down, the bearing will slide slightly, increasing the radius vector \vec{R} length from center. The z offset for the specified gradient can be calculated as follows. A tangent calculates t, using the radius of the plate in Y.

FIG. 16. Exemplary plate vector calculations.

$$Δ Z = Ry tan ( θ )$$

The top vector \vec{P}ₜ and bottom vector \vec{P}_{b} in this internal computation can point from the center of rotation about the x axis but corrected by the YZOffset as mentioned above.

$${\vec{P}}_{b} = [ \begin{matrix}- {R}_{y} \\ YZOffset - Z\end{matrix} ] , {\vec{P}}_{t} = [ \begin{matrix}{R}_{y} \\ YZOffset + Z\end{matrix} ]$$

d. Stage Y Offset to Apply Y Component of Gradient

The Y shift to apply the Y portion of the chip gradient is the z offset at the horizontal Y slider.

The X axis Z origin center (called Y_{L}, see the x axis discussion) can be subtracted to use the same Z reference position.

$$StageShiftY = {\vec{P}}_{t} \cdot Y - {Y}_{L / 2} / {S}_{y}$$

e. X, Z Correction for Translational Chip Shift

For Y, the plate does not translate. It just rotates about the Y origin at the X rail center.

$${\vec{P}}_{t r anslationaly} = 0$$

f. X, Z Correction for Rotational Chip Shift (from z Standoff Rotation)

Rotational shift x and z components:

$$Δ y = {S}_{0} sin ( θ )$$

$$Δ z = {S}_{0} - {S}_{0} cos ( θ )$$

$$Δ z = {S}_{0} ( 1 - cos ( θ ) )$$

$${\vec{P}}_{r o t a t ionaly} = [ \begin{matrix}Δ x \\ Δ z\end{matrix} ]$$

The same rotational correction can be used as in the X axis, by substituting the e in Y.

### 6. Final Outputs for an Input Desired Chip Gradient ∇Chip

a. Stage X and Stage Y to Apply Gradient

$${\vec{P}}_{s t a g e} = [ \begin{matrix}{\vec{p}}_{s t a g e {x}^{X}} \\ StageShiftY\end{matrix} ]$$

b. Combined X, Y, Z Chip Shift Output.

The final X, Y, Z chip shift vector can be the sum of the individual X and Y axis shift terms.

This shift can be applied to the imaging (e.g., raster imaging) in the x and y axis, as well as the Z pifoc window.

$${\vec{P}}_{c h i p x y z} = {\vec{P}}_{translationalx} + {\vec{P}}_{r o t a t ionalx} + 0 + {\vec{P}}_{r o t a tionaly}$$

### 7. Measuring, Monitoring, and Calibrating Gradient Slopes

a. The Applied Stage Gradients are Measured and Monitored Using a Laser Proximeter Focuser.

After chip leveling to the focal plane of the optics (for imaging a sample, such as a nucleic acid sample, e.g., a OGM sample), the error in such leveling can be measurable by taking z surface measurements of the chip in the corners (e.g., using the laser proximeter/focuser) and calculating the residual gradient error. This can be monitored during chip runs to measure accuracy and repeatability of the leveling operation.

b. Measured Errors in the Gradients Above can be Caused by Hardware Deviations from CAD Designs and are Corrected Via Gonio Ramp Slope Calibrations.

To impart a more accurate slope when leveling the chips, a gonio calibration can be applied to correct for hardware errors in the effective ramp slopes. This can be, for example, a linear calibration applied to the commanded gradient slope values. The relationship between the ramp slopes and applied slopes can be a non-linear function, and a solution can be an iterative method. To do this, a multiplier for both the X and Y gonio slopes can be calculated by applying commanded slopes and measuring actual slopes with the focuser as discussed in section a above.
//Sa Slope to Apply, —Sc Slope to Command Sr Slope Residual
//Sa=m Sc+b—Here m is the calibration linear coefficient
//Sc=(Sa−b)/m—Inverse to calculate slope to command

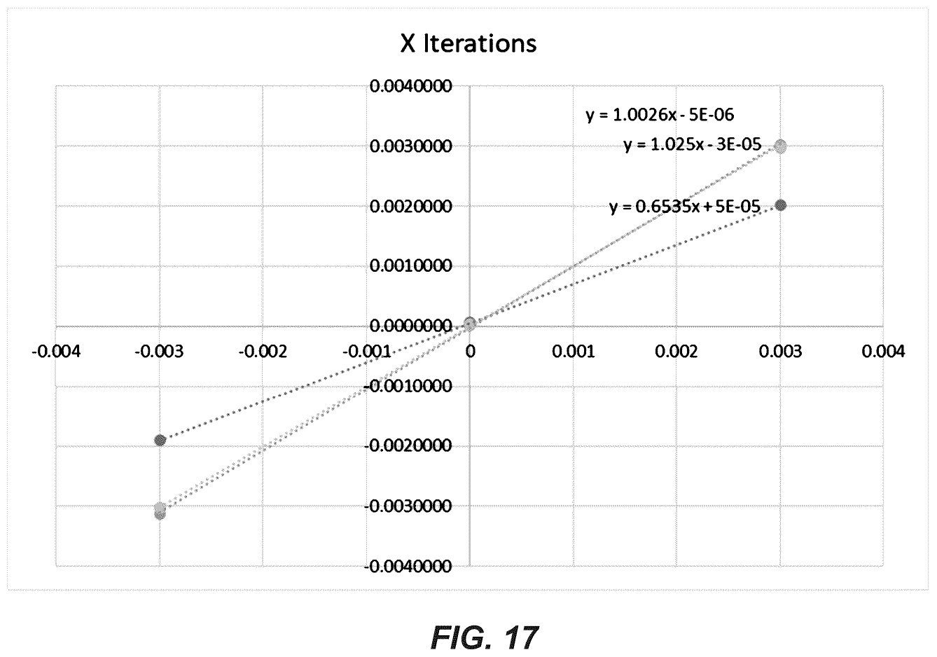

Using the measured residuals, linear regressions of commanded vs. actual gradient slopes can be used to calculate the gradient error. This can be done via an iterative approach, adjusting the calibration multiplier for both the x and y ramp slopes, and reapplying until the commanded vs. actual converges on the desired slope of 1.0 where commanded equals actual. At each iteration, the current ramp slope corrections, Mx and My, can be multiplied with the slope of the regression, as in the iterations plotted in FIG. 17, for a new Mx′, My′.

FIG. 17. Exemplary calibrating gonio slopes. Residuals added until desired slope of 1 achieved.

### Execution Environment

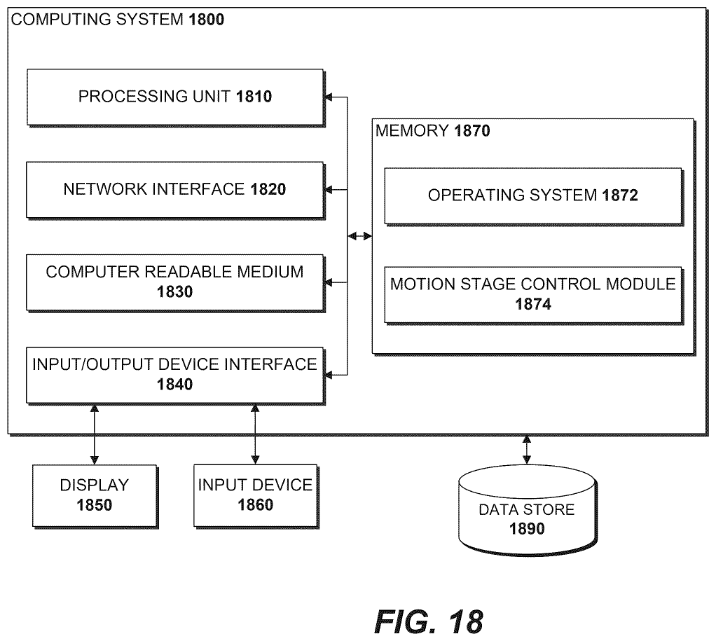

FIG. 18 depicts a general architecture of an example computing device 1800 that can be used in some embodiments to execute the processes and implement the features described herein. The general architecture of the computing device 1800 depicted in FIG. 18 includes an arrangement of computer hardware and software components. The computing device 1800 may include many more (or fewer) elements than those shown in FIG. 18. It is not necessary, however, that all of these generally conventional elements be shown in order to provide an enabling disclosure. As illustrated, the computing device 1800 includes a processing unit 1810, a network interface 1820, a computer readable medium drive 1830, an input/output device interface 1840, a display 1850, and an input device 1860, all of which may communicate with one another by way of a communication bus. The network interface 1820 may provide connectivity to one or more networks or computing systems. The processing unit 1810 may thus receive information and instructions from other computing systems or services via a network. The processing unit 1810 may also communicate to and from memory 1870 and further provide output information for an optional display 1850 via the input/output device interface 1840. The input/output device interface 1840 may also accept input from the optional input device 1860, such as a keyboard, mouse, digital pen, microphone, touch screen, gesture recognition system, voice recognition system, gamepad, accelerometer, gyroscope, or other input device.

The memory 1870 may contain computer program instructions (grouped as modules or components in some embodiments) that the processing unit 1810 executes in order to implement one or more embodiments. The memory 1870 generally includes RAM, ROM and/or other persistent, auxiliary or non-transitory computer-readable media. The memory 1870 may store an operating system 1872 that provides computer program instructions for use by the processing unit 1810 in the general administration and operation of the computing device 1800. The memory 1870 may further include computer program instructions and other information for implementing aspects of the present disclosure.

For example, in one embodiment, the memory 1870 includes a motion stage control module 1874 (e.g., a tip motion stage control module, a tilt motion stage control module, or a tip and tilt motion stage control module). The motion stage control module 1874 can determine the tip and/or tilt needed. Alternatively or additionally, the motion stage control module 1874 can cause the tip and/or the tilt of the motion stage to be adjusted (e.g., the motion stage control module 1874 can control the motion platform to adjust the tip and/or the tilt as needed). In addition, memory 1870 may include or communicate with the data store 1890 and/or one or more other data stores that store the final results (e.g., fluorescent images captured, and assembled maps) and/or intermediate results (e.g., tip and/or tilt adjustments needed, and fluorescent images captured) described herein.

### Optical Genome Mapping

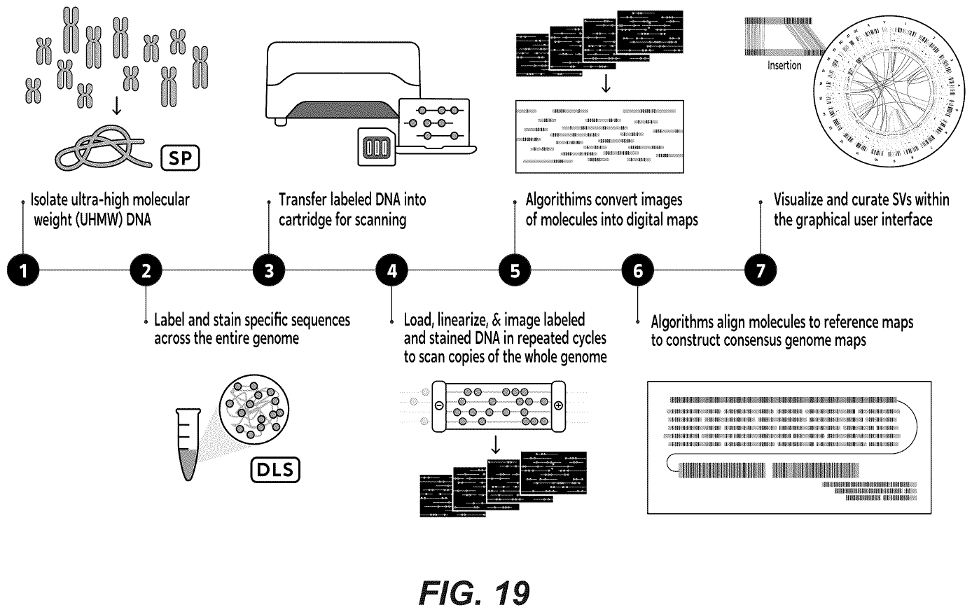

FIG. 19 illustrates a non-limiting exemplary workflow of optical genome mapping (OGM). The OGM workflow can start with mega-base size DNA isolation, e.g., 150 kbp or longer. A single enzymatic reaction can label the genome at a specific sequence motif occurring, e.g., approximately 15 times per 100 kbp in the human genome. The long, labeled DNA molecules can be linearized in nanochannel arrays (e.g., provided by a cartridge or chip, such as a Saphyr Chip® or newer, Bionano Genomics, Inc. (San Diego, CA)) and imaged in an automated manner by an OGM instrument (e.g., Saphyr® System or newer, Bionano Genomics, Inc. (San Diego, CA)). Samples being imaged can be placed precisely (e.g., using the designs, platforms, stages, and/or methods described herein) at the appropriate distance from the optics to produce focused images. The molecules can be assembled into local maps or whole genome maps. Changes in patterning or spacing of the labels can be detected, genome-wide, to call structural variants.

Optical Genome Mapping (OGM) is an imaging technology which evaluates the fluorescent labeling pattern of individual DNA molecules to perform an unbiased assessment of genome-wide structural variants down to, e.g., 500 base pairs (bp) in size, a resolution that far exceeds conventional cytogenetic approaches. OGM can rely on a specifically designed extraction protocol facilitating the isolation of high molecular weight (HMW) or ultra-high molecular weight (UHMW) DNA ultra-high molecular weight (UHMW) DNA. This protocol can, in some embodiments, utilize a paramagnetic disk purposed with trapping DNA for wash steps thereby reducing sheering forces present in standard column-based extraction methods. The result can be DNA fragments (or molecules) of about 150 kilobases (kbp) to megabases (Mbp) in size, about 5-10×longer than the average fragment size from conventional DNA isolations techniques. Referring to FIG. 6, DNA can be fluorescently labeled via covalent modification at a motif (which can be 4, 5, 6, 7, 8, 9, 10, or more nucleotides in length), such as a hexamer motif (e.g., the CTTAAG hexamer motif), generating genome-wide density of a number of labels per 100 kb in sequence specific patterns (e.g., approximately 14-17 labels per 100 kb, or 10, 11, 12, 13, 14, 15, 16, 17, 18, 19, 20, or more or fewer labels per 100 kb). Labeled DNA can be loaded on chips (e.g., silicon chips) composed of hundreds of thousands of parallel nanochannels where individual DNA molecules are linearized, imaged, and digitized. The specific labeling profile of individual DNA molecules, including spacing and pattern of hexamers labels, can be subsequently grouped based on similarity, producing about 500 kbp (or longer or shorter, such as 300 kbp, 400 kbp, 500 kbp, 600 kbp, 700 kbp, 800 kbp, 900 kbp, 1000 kbp) to megabase-sized consensus maps, which can be compared in silico to the expected labeling pattern of a reference genome (FIG. 6). This imaging technology converts DNA into a “barcode” whose labeling profile and characteristics can sensitively and specifically resolve copy number and structural variation without the need for sequence level data (FIG. 6). The quality of the DNA, including both size and labeling characteristics, as well as the number of images captured can influence genome-wide coverage. For example, each flow cell, which can accommodate a single specimen, can generate, for example, up to 5000 Gigabase pairs (Gbp) of raw data (or 3000 Gbp, 4000 Gbp, 5000 Gbp, 6000 Gbp, 7000 Gbp, 8000 Gbp, 9000 Gbp, 10000 Gbp, or more or less, of raw data), achieving a maximum theoretical genome-wide coverage of about 1250× (or 500×, 750×, 1000×, 1250×, 1500×, 1750×, 2000×, or more or less). Bioinformatics analyses can be performed. Example bioinformatics analysis can include: de novo structural variant analysis for typical germline assessments (e.g., greater than about 80×-coverage; requiring greater than about 400 Gbp data collection) or ‘Rare Variant Analysis (RVP)’ for somatic assessment down to a ˜5% variant allele fraction (e.g., greater than about 340× coverage; requiring greater than about 1500 Gbp data). Both algorithms facilitate the detection of a wide array of structural variants; from copy number gains/losses to balanced/unbalanced translocations and insertions to inversions.

Optical genome mapping (OGM) can be used to analyze large eukaryotic genomes and their structural features at a high resolution. OGM uses linearized strands of high molecular weight (HMW) or ultra-high molecular weight (UHMW) DNA that are far longer than the DNA sequences analyzed in current second- and third-generation sequencing methods, achieving average read lengths in excess of 200 kbp. The usage of long molecules in OGM can allow repetitive regions and other regions that are complicated to map to be spanned more easily than with short molecules. This leads to the creation of maps that may cover the whole arm of a chromosome and yet allow the detection of insertions and deletions as small as 500 bp (or longer or shorter, such as 300 kbp, 400 kbp, 500 kbp, 600 kbp, 700 kbp, 800 kbp, 900 kbp, 1000 kbp) other SVs may need to be 30 kbp (or 10 kbp, 20 k kbp, 30 kbp, 40 kbp, or 50 kbp)) or larger to be detectable. OGM can be used to, for example, detect the breakpoints of chromosomal translocations, for the diagnosis of facioscapulohumeral muscular dystrophy (FSHD). OGM may be used as a cytogenomic tool for prenatal diagnostics

Extraction/Isolation. UHMW DNA can be extracted for OGM, for example. UHMW DNA extraction can be done using isolation kits, such as kits from Bionano Genomics, Inc. (San Diego, CA). In some embodiments, DNA from approximately 1.5×10⁶ cells (or 1×10⁵, 1.5×10⁵, 2.5×10⁵, 5×10⁵, 7.5×10⁵, 1×10⁶, 1.5×10⁶, 2.5×10⁶, 5×10⁶, 7.5×10⁶, 1×10⁷ or more or fewer cells) can be extracted. The extraction can include immobilizing cells in agarose plugs and lysing the immunized cells by proteinase K; thereafter. The extraction can include washing, recovering, and quantifying the genomic DNA. Alternatively or additionally, the genomic DNA can be bound to a magnetic disk. Subsequently, the DNA can be washed, recovered, and quantified.

Labeling and Processing. A sufficient quantity of UHMW DNA (e.g., 250 ng, 500 ng, 750 ng, 1000 ng, 1250 ng, 1500 ng, 1750 ng, 2000 ng, or more UHMW DNA) can be labeled with a fluorophore. Such labeling can be done using a methyltransferase, such as the methyltransferase direct labeling enzyme (DLE-1) at the recognition motif of the methyltransferase, such as CTTAAG. This can generate a number of labels per 100 kbp (e.g., approximately 14-15 labels per 100 kbp, or 10, 11, 12, 13, 14, 15, 16, 17, 18, 19, 20, or more or less labels per kbp) when labeling human genomic DNA. In some embodiments, such labeling can be done using another enzyme (e.g., an endonuclease) at the recognition motif of the enzyme (e.g., GCTCTTCN of endonuclease Nt. BspQI).

Thereafter, the DNA can be dialyzed, its backbone stained, and finally the prepared DNA can be applied to flow cells (e.g., G1.2 flow cells from Bionano Genomics, Inc.) The flow cell can then be inserted into an OGM instrument, such as the Saphyr® instrument from Bionano Genomics, Inc. In the instrument, the DNA can be fed by electrophoresis into the nanochannels of the flow cell for linearization. DNA-filled nanochannels can be scanned using, for example, a fluorescence microscope. The captured images can be converted to electronic representations of the DNA molecules. The virtual DNA strands can then filtered and de novo assembled into maps (FIG. 6).

OGM Data Assembly. The data acquired with the OGM instrument can be processed. For example, the raw data can be filtered for a minimum length of 150 kbp (or 100 kbp, 125 kbp, 150 kbp, 175 kbp, 200 kbp, or more) and minimum of nine labels (or 5, 6, 7, 8, 9, 10, 11, 12, 13, 14, 15, or more labels) per molecule (or fragment). The filtered molecules can be assembled, e.g., with de novo assembly. The consensus maps of the molecules can be aligned to a reference genome sequence, such as the human reference genome GRCh38. Variants can be detected. Variants detection can be performed using, for example, a SV pipeline, comparing the maps to the aligned reference genome. There, patterns of markers from the maps deviating from the reference become apparent. Variants detections can be performed using, for example, a CNV pipeline,” which quantifies the mapped molecules and hence is able to detect gains and losses of several hundred kbp in size.

The results of the SV pipeline can then be augmented by, for example, a variant annotation pipeline, which adds quality metrics for the called variants and supplies their estimated frequency in the human population based on an internal database. The optional step of filtering based on the frequency of the SVs in the internal database may (or may not) be used in some implementations. The SVs can be detected or called. Automatic calling can be based on the confidence scores and sizes of the SVs (insertions and deletions: confidence >0, size >500 bp; inversions: confidence >0.7, size >30 kbp; duplications: confidence=−1, size >30 kbp; intrachromosomal translocations: confidence >0.3; interchromosomal translocations: confidence >0.65; CNV confidence >0.99, size >500 kbp). Additionally, each called SV can be required to be spanned by >5 strands of DNA.

The total amount of unfiltered DNA scanned by the OGM system can be, or be about, 750 Gbp, 800 Gbp, 850 Gbp, 900 Gbp, 916 Gbp, 925 Gbp, 950 Gbp, 1000 Gbp, 1250 Gbp, or more, per sample on average. An effective coverage of the reference can be, or can be greater than, 40×, 50×, 60×, 70×, 80×, 90×, or more, per sample. The effective coverage of the reference can be defined as the total length of filtered (>150 kbp) and aligned molecules divided by the length of the reference genome after de novo assembly

Further details regarding various aspects of OGM can be found in U.S. Pat. Nos. 11,359,244; 11,292,713; 11,291,999; 10,995,364; 10,844,424; 10,676,352; 10,669,586; 10,654,715; 10,435,739; 10,247,700; 10,000,804; 10,000,803; 9,845,238; 9,809,855; 9,804,122; 9,725,315; 9,536,041; 9,533,879; 9,310,376; 9,181,578; 9,061,901; 8,722,327; and 8,628,919; as well as published PCT Application Publication Nos. WO2020/005846; WO2016/036647; WO2015/134785; WO2015/130696; WO2015/126840; WO2015/017801; WO2014/200926; WO2014/130589; WO2014/123822; WO2013/036860; WO2012/054735; WO2011/050147; WO2011/038327 and WO2010/13532; the content of each of which is incorporated herein by reference in its entirety.

### Embodiments

Disclosed herein include embodiments of a method of positioning (or leveling or moving) a sample (e.g., adjusting the tip-axis and the tilt axis of the sample or TnT motion stage). A sample can be provided. The sample can be in a sample chip or cartridge. In some embodiments, a method of positioning a sample can comprise: providing a sample. The method can comprise: placing the sample, or the sample chip or cartridge, on (or onto or into) a sample carrier of a TnT motion stage of a motion platform of the present disclosure. The TnT motion stage can be on an x-y motion stage of the motion platform. The x-y motion stage can be on a base of the motion platform The method can include: determining a tip-tilt adjustment needed for the sample. The method can include: engaging a tip-axis adjustment paw (or tip adjustment engagement component) on the base of the motion platform with a tip-axis adjustment notch (or complementary tip adjustment engagement component) of the TnT motion stage. The method can include: moving an x-y motion stage of the motion platform along one axis of the x-axis and the y-axis, thereby changing the tip of the TnT motion stage, based on the tip-tilt adjustment needed. The method can include engaging a tilt-axis adjustment paw (or tilt adjustment engagement component) on the base of the motion platform with a tilt-axis adjustment notch (or complementary tilt adjustment engagement component) of the TnT motion stage. The method can include: moving the x-y motion stage along the one axis of the x-axis and the y-axis, thereby changing the tilt of the TnT motion stage, based on the tip-tilt adjustment needed.

In some embodiments, a method of positioning a sample comprises: providing a sample. The sample can be in a sample chip or cartridge. The method can comprise: placing the sample, or the sample chip or cartridge, on (or onto or into) a sample carrier of a tip and tilt (TnT) motion stage of a motion platform of the present disclosure. The TnT motion stage can be on an x-y motion stage of the motion platform. The x-y motion stage can be on a base of the motion platform. The method can comprise, iteratively, determining a tip-tilt adjustment needed for the sample. The iterative process can include: engaging a tip-axis adjustment paw (or tip adjustment engagement component) on the base of the motion platform with a tip-axis adjustment notch (or complementary tip adjustment engagement component) of the TnT motion stage. The iterative process can include: moving an x-y motion stage of the motion platform along one axis of the x-axis and the y-axis, thereby changing the tip of the TnT motion stage, based on the tip-tilt adjustment needed. The iterative process can include: engaging a tilt-axis adjustment paw (or tilt adjustment engagement component) on the base of the motion platform with a tilt-axis adjustment notch (or complementary tilt adjustment engagement component) of the TnT motion stage. The iterative process can include: moving the x-y motion stage along the one axis of the x-axis and the y-axis, thereby changing the tilt of the TnT motion stage, based on the tip-tilt adjustment needed.

In some embodiments, the iterative process comprises: determining a chip gradient. The iterative process can comprise: engaging a tip-axis adjustment paw on the base of the motion platform with a tip-axis adjustment notch of the TnT motion stage. The iterative process can comprise: moving an x-y motion stage of the motion platform along one axis of the x-axis and the y-axis, thereby changing the tip of the TnT motion stage, based on the chip gradient. The iterative process can comprise: engaging a tilt-axis adjustment paw on the base of the motion platform with a tilt-axis adjustment notch of the TnT motion stage. The iterative process can comprise: moving the x-y motion stage along the one axis of the x-axis and the y-axis, thereby changing the tilt of the TnT motion stage, based on the chip gradient adjustment needed. The iterative process can comprise: determining a FOV gradient. The iterative process can comprise: engaging a tip-axis adjustment paw on the base of the motion platform with a tip-axis adjustment notch of the TnT motion stage. The iterative process can comprise: moving an x-y motion stage of the motion platform along one axis of the x-axis and the y-axis, thereby changing the tip of the TnT motion stage, based on the FOV gradient. The iterative process can comprise: engaging a tilt-axis adjustment paw on the base of the motion platform with a tilt-axis adjustment notch of the TnT motion stage. The iterative process can comprise: moving the x-y motion stage along the one axis of the x-axis and the y-axis, thereby changing the tilt of the TnT motion stage based on the FOV gradient adjustment needed.

In some embodiments, a method of positioning a sample comprises: (a) providing a sample. The sample can be in a sample chip or cartridge. The method can comprise: (b) placing the sample, or the sample chip or cartridge, on (or onto or into) a sample carrier of a tip and tilt (TnT) motion stage of a motion platform of the present disclosure. The TnT motion stage can be on an x-y motion stage of the motion platform. The x-y motion stage can be on a base of the motion platform. The method can comprise: (c1) determining a chip gradient of the sample. The method can comprise: (d1) performing one or more steps of the following based on the chip gradient in step (d1). The method can comprise: engaging a tip-axis adjustment paw (or tip adjustment engagement component) on the base of the motion platform with a tip-axis adjustment notch (or complementary tip adjustment engagement component) of the TnT motion stage. The method can comprise: moving an x-y motion stage of the motion platform along one axis of the x-axis and the y-axis, thereby changing the tip of the TnT motion stage. The method can comprise: engaging a tilt-axis adjustment paw (or tilt adjustment engagement component) on the base of the motion platform with a tilt-axis adjustment notch (or complementary tilt adjustment engagement component) of the TnT motion stage. The method can comprise: moving the x-y motion stage along the one axis of the x-axis and the y-axis, thereby changing the tilt of the TnT motion stage. In some embodiments, the method can comprise: (c2) determining a field of view (FOV) gradient. The method can comprise: (d2) performing one or more steps of (d1) based on the FOV gradient.

In some embodiments, a method of positioning a sample comprises: providing a sample. The sample can be in a sample chip or cartridge. The method can include: placing the sample, or the sample chip or cartridge) on (or onto or into) a sample carrier of a tip and tilt (TnT) motion stage of a motion platform of the present disclosure. The TnT motion stage can be on an x-y motion stage of the motion platform. The x-y motion stage can be on a base of the motion platform. The method can include: determining a chip gradient of the sample. The method can include: engaging a tip-axis adjustment paw (or tip adjustment engagement component) on the base of the motion platform with a tip-axis adjustment notch (or complementary tip adjustment engagement component) of the TnT motion stage and moving an x-y motion stage of the motion platform along one axis of the x-axis and the y-axis, thereby changing the tip of the TnT motion stage, based on the chip gradient. The method can include: engaging a tilt-axis adjustment paw (or tilt adjustment engagement component) on the base of the motion platform with a tilt-axis adjustment notch (or complementary tilt adjustment engagement component) of the TnT motion stage and moving the x-y motion stage along the one axis of the x-axis and the y-axis, thereby changing the tilt of the TnT motion stage, based on the chip gradient. The method can include: determining a field of view (FOV) gradient. The method can include: engaging the tip-axis adjustment paw with the tip-axis adjustment notch and moving an x-y motion stage of the motion platform along the one axis of the x-axis and the y-axis, thereby changing the tilt of the TnT motion stage, based on the FOV gradient. The method can include: engaging the tilt-axis adjustment paw with the tilt-axis adjustment notch and moving the x-y motion stage along the one axis of the x-axis and the y-axis, thereby changing the tilt of the TnT motion stage, based on the FOV gradient.

In some embodiments, a method of positioning a sample can comprise: providing a sample. The method can comprise: placing the sample, or the sample chip or cartridge, on (or onto or into) a sample carrier of a TnT motion stage of a motion platform of the present disclosure. The method can comprise: determining a tip adjustment needed. The method can comprise: determining a tilt adjustment needed. In some embodiments, the method comprises: determining a tip adjustment needed, a tilt adjustment needed, or a combination thereof. The method can comprise: engaging the tip-axis adjustment paw (or tip adjustment engagement component) with the tip-axis adjustment notch (or complementary tip adjustment engagement component). The method can comprise: moving the x-y motion stage along one axis (e.g., the y-axis) of the x-axis and the y-axis based on the tip adjustment needed. This can result in changing the tip of the TnT motion stage. The method can include: engaging the tilt-axis adjustment paw (or tilt adjustment engagement component) with the tilt-axis adjustment notch (or tilt complementary adjustment engagement component). The method can include: moving the x-y motion stage along the one axis (e.g., the y-axis) of the x-axis and the y-axis based on the tilt adjustment needed. This can result in changing the tilt of the TnT motion stage. Prior to engaging the tilt-axis adjustment paw with the tilt-axis adjustment notch, the method can include: disengaging the tip-axis adjustment paw with the tip-axis adjustment notch

In some embodiments, engaging the tip-axis adjustment paw with the tip-axis adjustment notch and moving the x-y motion stage along the one axis of the x-axis and the y-axis occurs before engaging the tilt-axis adjustment paw with the tilt-axis adjustment notch and moving the x-y motion stage along the one axis of the x-axis and the y-axis, thereby changing the tilt of the TnT motion stage. Prior to engaging the tilt-axis adjustment paw with the tilt-axis adjustment notch, the method can include: disengaging the tip-axis adjustment paw with the tip-axis adjustment notch. In some embodiments, engaging the tip-axis adjustment paw with the tip-axis adjustment notch and moving the x-y motion stage along the one axis of the x-axis and the y-axis occurs after engaging the tilt-axis adjustment paw with the tilt-axis adjustment notch and moving the x-y motion stage along the one axis of the x-axis and the y-axis, thereby changing the tilt of the TnT motion stage. Prior to engaging the tip-axis adjustment paw with the tip-axis adjustment notch, the method can include: disengaging the tilt-axis adjustment paw with the tilt-axis adjustment notch.

In some embodiments, the first-axis comprises the tip-axis. In some embodiments, the first-axis comprises the tilt-axis. In some embodiments, the method further comprises: determining a second-axis adjustment needed. The method can comprise: engaging the second-axis adjustment engagement component with the second-axis complementary adjustment engagement component. The method can comprise: moving the x-y motion stage along the one axis of the x-axis and the y-axis, thereby changing the second-axis of the motion stage, based on the first-axis adjustment needed. In some embodiments, the second-axis comprises the tip-axis. In some embodiments, the second-axis comprises the tilt-axis.

In some embodiments, the tip-tilt adjustment comprises a chip gradient (e.g., qc), a leveling gradient (e.g., φ_{L}), and/or a field of view (FOV) gradient (e.g., φ_{F}). In some embodiments, the tip-tilt adjustment comprises (i) a stage X offset or a X component of gradient and/or (ii) a stage Y offset or a Y component of gradient. The tip-tilt adjustment can comprise a tip adjustment and a tilt adjustment. The tip-adjustment can comprise a stage X offset (e.g., \vec{P}_{stagex}X) or a X component of gradient. The tilt-adjustment can comprise a stage Y offset (e. g., StageShiftY) or a Y component of gradient. In some embodiments, determining the tip-tilt adjustment comprises: determining a X, Y, Z shift vector (e.g., \vec{P}_{chipxyz}=\vec{P}ₜᵣₐₙₛₗₐₜᵢₒₙₐₗₓ+\vec{P}ᵣₒₜₐₜᵢₒₙₐₗₓ+0+\vec{P}_{rotationaly}).

In some embodiments, the method comprises: changing the x-y position of the motion stage. Changing the x-y position of the motion stage can occur before changing the tip of the TnT motion stage and/or the tilt of the TnT motion stage. Changing the x-y position of the motion stage can occur after changing the tip of the TnT motion stage and/or the tilt of the TnT motion stage.

In some embodiments, the sample is in a sample chip or cartridge. In some embodiments, placing the sample on the sample carrier can comprise placing the sample chip or cartridge on the sample carrier.

In some embodiments, a method of positioning (or leveling or moving) a sample comprises: providing a sample. The sample can be in a sample chip or cartridge. The method can include: placing the sample, or the sample chip or cartridge, on (or onto or into) a sample carrier of a tip motion stage of a motion platform. The method can include: determining a tip adjustment needed. The method can include: engaging the tip-axis adjustment engagement component with the tip-axis complementary adjustment engagement component. The method can include: moving the x-y motion stage along one axis of the x-axis and the y-axis based on the tip adjustment needed. This can result in changing the tip of the tip motion stage.

In some embodiments, a method of positioning (or leveling or moving) a sample comprises: providing a sample. The sample can be in a sample chip or cartridge. The method can include: placing the sample, or the sample chip or cartridge, on (or onto or into) a sample carrier of a tilt motion stage of a motion platform. The method can include: determining a tilt adjustment needed. The method can include: engaging the tilt-axis adjustment engagement component with the tilt-axis complementary adjustment engagement component. The method can include: moving the x-y motion stage along one axis of the x-axis and the y-axis based on the tilt adjustment needed. This can result in changing the tilt of the tilt motion stage.

In some embodiments, a method of positioning (or leveling or moving) a sample comprises: providing a sample. The sample can be in a sample chip or cartridge. The method can include: placing the sample, or the sample chip or cartridge, on (or onto or into) a sample carrier of a motion stage of a motion platform. The method can include: determining a first-axis adjustment needed. The method can include: engaging the first-axis adjustment engagement component with the first-axis complementary adjustment engagement component. The method can include: moving the x-y motion stage along one axis of the x-axis and the y-axis based on the first-axis adjustment needed. This can result in changing the first-axis of the motion stage.

Disclosed herein include methods of imaging a sample. In some embodiments, a method of imaging a sample comprising: positioning (e.g., adjusting the tip-axis and/or tilt-axis) a sample as described herein. The sample can be on (or in) a sample carrier of a TnT motion stage of a motion platform of the present disclosure. Positioning the sample can include adjusting the tip-axis and/or tilt-axis of the TnT motion stage as described herein. The method can include: rastering, using the x-y motion stage, to different positions along the x-axis and/or the y-axis. The method can include capturing images of the sample at the different positions. In some embodiments, the tip-axis and/or tilt-axis of the sample (or the TnT motion stage) can be adjusted after a number of images of the sample are captured at different positions. In some embodiments, the tip-axis and/or tilt-axis of the sample (or the TnT motion stage) may not need to be adjusted.

In some embodiments, the sample comprises an optical genome mapping (OGM) sample. In some embodiments, the sample comprises nucleic acids. The nucleic acids can comprise deoxyribonucleic acid (DNA). The nucleic acids can comprise the nucleic acids comprise genomic DNA. The nucleic acids can comprise fragmented genomic DNA. The nucleic acids can comprise ribonucleic acids (RNA). The nucleic acids can comprise DNA derived (e.g., reverse transcribed) from DNA or RNA. In some embodiments, the sample comprises labeled nucleic acids, optionally wherein the sample comprises fluorescently labeled nucleic acids.

In some embodiments, a motion platform can comprise: a base. The motion platform can comprise: an x-y motion stage. The x-y motion stage can be on (e.g., attached to, such as securely attached to) the base. The motion platform can comprise: a tip-axis adjustment pawl (or a tip-axis adjustment engagement component) on the base. The motion platform can comprise: a tilt-axis adjustment pawl (or a tilt-axis adjustment engagement component) on (e.g., attached to, such as securely attached to) the base. The motion platform can comprise: a tip and tilt (TnT) motion stage. The TnT motion stage can be on the x-y motion stage. The TnT motion stage can comprise: two tip-axis goniometers with different slopes (e.g., 3.6° and −3.6° respectively as illustrated in FIG. 2) relative to one axis (e.g., x-axis) of the x-axis and the y-axis (e.g., of the motion platform or the motion stage). The TnT motion stage can comprise: a bearing, such as a tilt-axis slanted linear bearing (e.g., slanted relative to the plane of the platform and/or the x-y motion stage). The bearing can be a recirculating linear bearing. The bearing can be a non-recirculating linear bearing. The TnT motion stage can comprise: a tip-axis adjustment notch (or a tip-axis complementary adjustment engagement component). The TnT motion stage can comprise: a tilt-axis adjustment notch (or a tilt-axis complementary adjustment engagement component). In some embodiments, the TnT motion stage can comprise: a sample carrier. In some embodiments, when the tip-axis adjustment pawl is engaged with the tip-axis adjustment notch, a movement of the x-y motion stage along the one axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

In some embodiments, a motion platform can comprise: a base. The motion platform can comprise: an x-y motion stage on the base. The motion platform can comprise: a tip-axis adjustment pawl (or a tip-axis adjustment engagement component) on the base. The motion platform can comprise: a tip motion stage. The tip motion stage can be on the x-y motion stage. The tip motion stage can comprise: one or more (e.g., 2) tip-axis goniometers with different slopes relative to one axis of the x-axis and the y-axis. The tip motion stage can comprise: a tip-axis adjustment notch. In some embodiments, when the tip-axis adjustment pawl is engaged with the tip-axis adjustment notch, a movement of the x-y motion stage along the one axis results in a movement of the tip motion stage along the one or more tip-axis goniometers. This can result in a change in the tip of the tip motion stage. In some embodiments, the tip motion stage can comprise: a sample carrier.

In some embodiments, the motion platform further comprises: a tilt-axis adjustment pawl (or a tilt-axis adjustment engagement component) on the base. The tip motion stage can be a tip and tilt (TnT) motion stage. The TnT motion stage can further comprise: a tilt-axis slanted linear bearing. The TnT motion stage can further comprise: a tilt-axis adjustment notch (or a tilt-axis complementary adjustment engagement component). In some embodiments, when the tilt-axis adjustment pawl is engaged with the tilt-axis adjustment notch, a movement of the x-y motion stage along the one axis results in a movement of the TnT motion stage along the tilt-axis slanted linear bearing. This can result in a change in the tilt of the TnT motion stage.

In some embodiments, a motion platform can comprise: a base. The motion platform can comprise: an x-y motion stage on the base. The motion platform can comprise: a tilt-axis adjustment pawl (or a tilt-axis adjustment engagement component) on the base. The motion platform can comprise: a tilt motion stage. The tilt motion stage can be on the x-y motion stage. The tilt motion stage can comprise: a tilt-axis slanted linear bearing. The tilt motion stage can comprise: a tilt-axis adjustment notch (or a tilt-axis complementary adjustment engagement component). In some embodiments, when the tilt-axis adjustment pawl is engaged with the tilt-axis adjustment notch, a movement of the x-y motion stage along the one axis results in a movement of the tilt motion stage along the tilt-axis slanted linear bearing. This can result in a change in the tilt of the tilt motion stage. In some embodiments, the tilt motion stage can comprise: a sample carrier.

In some embodiments, the motion platform further comprises: a tip-axis adjustment pawl on the base. The tilt motion stage can be a tip and tilt (TnT) motion stage. The TnT motion stage can further comprise: one or more (e.g., 2) tip-axis goniometers with different slopes relative to one axis of the x-axis and the y-axis. The TnT motion stage can further comprise: a tip-axis adjustment notch (or a tip-axis complementary adjustment engagement component). In some embodiments, when the tip-axis adjustment pawl is engaged with the tip-axis adjustment notch, a movement of the x-y motion stage along the one axis results in a movement of the TnT motion stage along the one or more tip-axis goniometers. This can result in a change in the tip of the TnT motion stage.

In some embodiments, a motion platform can comprise: a base. The motion platform can comprise: an x-y motion stage. The x-y motion stage can be on (e.g., attached to, such as securely attached to) the base. The motion platform can comprise: a first-axis (e.g., a tip-axis or a tilt-axis) adjustment engagement component (e.g., a pawl or a notch) on (e.g., attached to, such as securely attached to) the base. The motion platform can comprise: a motion stage (e.g., a tip motion stage, a tilt motion stage, or a tip and tilt (TnT) motion stage). The second motion stage can be on the x-y motion stage. The motion platform can comprise: a first-axis goniometer or bearing. The motion platform can comprise: a first-axis complementary adjustment engagement component (e.g., a notch or a pawl), The first-axis adjustment engagement component and the first-axis complementary adjustment engagement component can engage (or be in engagement, such as secure engagement) with each other. For example, the first-axis adjustment engagement component and the first-axis complementary engagement component can be a pawl and a notch and can be in engagement (e.g., secure engagement) with each other. When the first-axis adjustment component is engaged with the first-axis complementary adjustment engagement component, a movement of the x-y motion stage along one axis (e.g., x-axis) of the x-axis and the y-axis can result. In a movement of the second motion stage along the first-axis goniometer or bearing. This can result in a change in the first-axis (e.g., tip-axis) of the second motion stage. In some embodiments, the motion platform can comprise: a sample carrier.

In some embodiments, the motion platform can further comprise a second-axis (e.g., a tilt-axis or a tip-axis) adjustment engagement component (e.g., a pawl or a notch). The second motion stage can further comprise: a second-axis goniometer or bearing. The second motion stage can further comprise: a second-axis complementary adjustment engagement component (e.g., a notch or a pawl). The second-axis adjustment engagement component and the second-axis complementary adjustment engagement component can engage (or be in engagement, such as secure engagement) with each other. For example, the second-axis adjustment engagement component and the second-axis complementary engagement component can be a pawl and a notch and can be in engagement (e.g., secure engagement) with each other. When the second-axis adjustment engagement component is engaged with the second-axis complementary adjustment engagement component, a movement of the x-y motion stage along the one axis can result in a movement of the second motion stage along the second-axis goniometer or bearing. This can result in a change in the second-axis (e.g., tilt-axis) of the second motion stage.

In some embodiments, the first-axis is the tip-axis. The second-axis can be the tilt-axis. In some embodiments, the first-axis is the tilt-axis. The second-axis can be the tip-axis. In some embodiments, the first-axis goniometer can comprise one or more (e.g., 2) first-axis goniometers. In some embodiments, the first-axis bearing comprises a first-axis slanted linear bearing.

In some embodiments, the motion platform comprises an x-axis motor on (e.g., attached to, such as securely attached to) the base. The motion platform can comprise a y-axis motor on (e.g., attached to, such as securely attached to) the base. The x-axis motor can move the x-y motion stage along the x-axis. The y-axis motor can move the x-y motion stage along the y-axis. The motion platform can comprise no additional motor other than the x-axis motor and the y-axis motor for changing the tip and/or tilt of the TnT motion stage. In some embodiments, the x-axis motor is a servomotor. The y-axis motor can be a servomotor.

In some embodiments, the tip-axis adjustment pawl and the tilt-axis adjustment pawl point in the opposite directions. In some embodiments, the tip-axis adjustment notch and the tilt-axis adjustment notch point in the opposite directions. In some embodiments, the tip-axis adjustment pawl and the tilt-axis adjustment pawl are elevated from the base. In some embodiments, tip-axis adjustment pawl and the tilt-axis adjustment pawl are at different heights relative to the base. The tilt-axis adjustment notch and the tip-axis adjustment notch can be at different heights relative to the base. In some embodiments, the tip-axis adjustment pawl and the tilt-axis adjustment pawl are at an identical height relative to the base. The tilt-axis adjustment notch and the tip-axis adjustment notch can be at an identical height relative to the base.

In some embodiments, the TnT motion stage comprises no motor. In some embodiments, the TnT motion stage is in contact with the x-y motion stage via the two tip-axis goniometers and the tilt-axis slanted linear bearing. In some embodiments, the TnT motion stage is in contact with the x-y motion stage only via the two tip-axis goniometers and the tilt-axis slanted linear bearing.

In some embodiments, the TnT motion stage comprises no motor. In some embodiments, the TnT motion stage is in contact with the x-y motion stage via the one or more first-axis goniometers and the second-axis bearing. In some embodiments, the TnT motion stage is in contact with the x-y motion stage only via the one or more first-axis goniometers and the second-axis bearing.

In some embodiments, the two tip-axis goniometers have different slopes relative to the x-axis (or the y-axis). In some embodiments, the angle of the slope of the other of the two tip-axis goniometers can be, be about, be at least, be at least about, be at most, or be at most about, ±1°, ±1.1°, ±1.2°, ±1.3°, ±1.4°, ±1.5°, ±1.6°, ±1.7°, ±1.8°, ±1.9°, ±2°, ±2.1°, ±2.2°, ±2.3°, ±2.4°, ±2.5°, ±2.6°, ±2.7°, ±2.8°, ±2.9°, ±3.0°, ±3.1°, ±3.2°, ±3.3°, ±3.4°, ±3.5°, ±3.6°, ±3.7°, ±3.8°, ±3.9°, ±4°, ±4.1°, ±4.2°, ±4.3°, ±4.4°, ±4.5°, ±4.6°, ±4.7°, ±4.8°, ±4.9°, ±5°, ±5.1°, ±5.2°, ±5.3°, ±5.4°, ±5.5°, ±5.6°, ±5.7°, ±5.8°, ±5.9°, ±6°, ±6.1°, 16.2°, ±6.3°, ±6.4°, 16.5°, ±6.6°, ±6.7°, ±6.8°, ±6.9°, ±7°, ±7.1°, ±7.2°, ±7.3°, ±7.4°, ±7.5°, ±7.6°, ±7.7°, ±7.8°, ±7.9°, ±8°, ±8.1°, ±8.2°, ±8.3°, ±8.4°, ±8.5°, ±8.6°, ±8.7°, ±8.8°, ±8.9°, ±9°, ±9.1.°, ±9.2°, ±9.3°, ±9.4°, ±9.5°, ±9.6°, ±9.7°, ±9.8°, ±9.9°, ±10°, or a number or a range between any two of these values. In some embodiments, the slopes of the two tip-axis goniometers have different absolute angles. In some embodiments, the slopes of the two tip-axis goniometers have an identical absolute angle. In some embodiments, the absolute angle of the slope of one or each of the two tip-axis goniometers is about 3.6° (see FIG. 2 for an illustration). In some embodiments, the absolute angle of the slope of one or each of the two tip-axis goniometers can be, be about, be at least, be at least about, be at most, or be at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, one or each of the two tip-axis goniometers is at or adjacent to a side surface (e.g., a vertical surface relative to the platform or the x-y motion stage) of the TnT motion platform. The two tip-axis goniometers can be at or adjacent to the same side surface or different side surfaces of the TnT motion platform. In some embodiments, one or each of the two tip-axis goniometers comprises a journal and a slanted pin. The material of the journal can comprise bronze. The material of the pin can comprise stainless steel. In some embodiments, one or each of the two tip-axis goniometers comprises a magnet. The magnet can retain contact between the journal and the slanted pin.

In some embodiments, the tilt-axis slanted linear bearing comprises a linear bearing carriage and a slanted linear bearing rail. In some embodiments, the linear bearing motion angle of the tilt-axis slanted linear bearing is about 3.4° (see FIGS. 3-4 for an illustration). In some embodiments, the linear bearing motion angle of the tilt-axis slanted linear bearing is, is about, is at least, is at least about, is at most, or is at most about, ±1°, ±1.1°, ±1.2°, ±1.3°, ±1.4°, ±1.5°, ±1.6°, ±1.7°, ±1.8°, ±1.9°, ±2°, ±2.1°, ±2.2°, ±2.3°, ±2.4°, ±2.5°, ±2.6°, ±2.7°, ±2.8°, ±2.9°, ±3.0°, ±3.1°, ±3.2°, ±3.3°, ±3.4°, ±3.5°, ±3.6°, ±3.7°, ±3.8°, ±3.9°, ±4°, ±4.1°, ±4.2°, ±4.3°, ±4.4°, ±4.5°, ±4.6°, ±4.7°, ±4.8°, ±4.9°, ±5°, ±5.1°, ±5.2°, ±5.3°, ±5.4°, ±5.5°, ±5.6°, ±5.7°, ±5.8°, ±5.9°, ±6°, ±6.1°, ±6.2°, ±6.3°, ±6.4°, ±6.5°, ±6.6°, ±6.7°, ±6.8°, ±6.9°, ±7°, ±7.1°, ±7.2°, ±7.3°, ±7.4°, ±7.5°, ±7.6°, ±7.7°, ±7.8°, ±7.9°, ±8°, ±8.1°, ±8.2°, ±8.3°, ±8.4°, ±8.5°, ±8.6°, ±8.7°, ±8.8°, ±8.9°, ±9°, ±9.1.°, ±9.2°, ±9.3°, ±9.4°, ±9.5°, ±9.6°, ±9.7°, ±9.8°, ±9.9°, ±10°, or a number or a range between any two of these values. In some embodiments, the absolute value of the linear bearing motion angle of the tilt-axis slanted linear bearing is, is about, is at least, is at least about, is at most, or is at most about, 1°, 1.1°, 1.2°, 1.3°, 1.4°, 1.5°, 1.6°, 1.7°, 1.8°, 1.9°, 2°, 2.1°, 2.2°, 2.3°, 2.4°, 2.5°, 2.6°, 2.7°, 2.8°, 2.9°, 3.0°, 3.1°, 3.2°, 3.3°, 3.4°, 3.5°, 3.6°, 3.7°, 3.8°, 3.9°, 4°, 4.1°, 4.2°, 4.3°, 4.4°, 4.5°, 4.6°, 4.7°, 4.8°, 4.9°, 5°, 5.1°, 5.2°, 5.3°, 5.4°, 5.5°, 5.6°, 5.7°, 5.8°, 5.9°, 6°, 6.1°, 6.2°, 6.3°, 6.4°, 6.5°, 6.6°, 6.7°, 6.8°, 6.9°, 7°, 7.1°, 7.2°, 7.3°, 7.4°, 7.5°, 7.6°, 7.7°, 7.8°, 7.9°, 8°, 8.1°, 8.2°, 8.3°, 8.4°, 8.5°, 8.6°, 8.7°, 8.8°, 8.9°, 9°, 9.1.°, 9.2°, 9.3°, 9.4°, 9.5°, 9.6°, 9.7°, 9.8°, 9.9°, 10°, or a number or a range between any two of these values.

In some embodiments, the tilt-axis slanted linear bearing is at or adjacent a (or a second) side surface (e.g., a vertical surface relative to the platform or the x-y motion stage) of the TnT motion platform. In some embodiments, the TnT motion stage comprises a radial bearing in contact with a radial bearing rail. A material of the radial bearing can comprise stainless steel. A material of the radial bearing rail can comprise stainless steel. The radial bearing rail can be co-planar with the x-axis. In some embodiments, the TnT motion stage comprises at least one magnet (e.g., 2 magnets) which retains contact between the radial bearing and the radial bearing rail. In some embodiments, the radial bearing is at or adjacent to a side surface (or the second side surface). In some embodiments, the TnT motion stage comprises 8 side surfaces. In some embodiments, the TnT motion stage comprises 3, 4, 5, 6, 7, 8, 9, 10, 11, 12, 13, 14, 15, or a number or a range between any two of these values, side surfaces.

In some embodiments, when the tip-axis adjustment pawl is engaged with the tip-axis adjustment notch, the tilt-axis adjustment pawl is not engaged with the tilt-axis adjustment notch. When the tilt-axis adjustment pawl is engaged with the tilt-axis adjustment notch, the tip-axis adjustment pawl may be not engaged with the tip-axis adjustment notch. In some embodiments, when the tip-axis adjustment pawl is engaged with the tip-axis adjustment notch, a movement of the x-y motion stage along the x-axis results in a movement of the TnT motion stage along the two tip-axis goniometers. This can result in a change in the tip of the TnT motion stage. In some embodiments, when the tilt-axis adjustment pawl is engaged with the tilt-axis adjustment notch, a movement of the x-y motion stage along the x-axis results in a movement of the TnT motion stage along the tilt-axis slanted linear bearing. This can result in a change in the tilt of the TnT motion stage.

In some embodiments, when the TnT motion stage is moved along the y-axis to the edge of its travel in one direction of the y-axis, the tip-axis adjustment pawl engages with the tip-axis adjustment notch. When the TnT motion stage is moved along the y-axis to the edge of its travel in the other direction of the y-axis, the tilt-axis adjustment pawl can engage with the tilt-axis adjustment notch.

In some embodiments, an instrument comprises the motion platform. The instrument can comprise: a sensor (e.g., below or above the motion platform). The instrument can comprise: optics (e.g., below or above the motion platform). The instrument can comprise a fluorescent imaging system, such as an optical genome mapping (OGM) system. In some embodiments, the base of the motion platform is fixed in position within the motion platform. In some embodiments, the motion platform is suspended within the instrument.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A motion platform comprising:
a base;
an x-y motion stage on the base;
a tip-axis adjustment pawl and a tilt-axis adjustment pawl on the base;
a tip and tilt (TnT) motion stage on the x-y motion stage and comprising:
a sample carrier,
two tip-axis goniometers with different slopes relative to one axis of the x-axis and the y-axis, and
a tilt-axis slanted linear bearing,
a tip-axis adjustment notch, and
a tilt-axis adjustment notch,
wherein when the tip-axis adjustment pawl is engaged with the tip-axis adjustment notch, a movement of the x-y motion stage along the one axis results in a movement of the TnT motion stage along the two tip-axis goniometers, thereby resulting in a change in the tip of the TnT motion stage, and
wherein when the tilt-axis adjustment pawl is engaged with the tilt-axis adjustment notch, a movement of the x-y motion stage along the one axis results in a movement of the TnT motion stage along the tilt-axis slanted linear bearing, thereby resulting in a change in the tilt of the TnT motion stage.

2. A motion platform comprising:
a base;
an x-y motion stage on the base;
a tip-axis adjustment pawl on the base;
a tip motion stage on the x-y motion stage and comprising:
a sample carrier,
one or more tip-axis goniometers with different slopes relative to one axis of the x-axis and the y-axis, and
a tip-axis adjustment notch, and
wherein when the tip-axis adjustment pawl is engaged with the tip-axis adjustment notch, a movement of the x-y motion stage along the one axis results in a movement of the tip motion stage along the one or more tip-axis goniometers, thereby resulting in a change in the tip of the tip motion stage.

3. The motion platform of claim 2,
wherein the motion platform further comprises: a tilt-axis adjustment pawl on the base,
wherein the tip motion stage is a tip and tilt (TnT) motion stage further comprises:
a tilt-axis slanted linear bearing, and
a tilt-axis adjustment notch,
wherein when the tilt-axis adjustment pawl is engaged with the tilt-axis adjustment notch, a movement of the x-y motion stage along the one axis results in a movement of the TnT motion stage along the tilt-axis slanted linear bearing, thereby resulting in a change in the tilt of the TnT motion stage.

4. A motion platform comprising:
a base;
an x-y motion stage on the base;
a tilt-axis adjustment pawl on the base;
a tilt motion stage on the x-y motion stage and comprising:
a sample carrier,
a tilt-axis slanted linear bearing, and
a tilt-axis adjustment notch,
wherein when the tilt-axis adjustment pawl is engaged with the tilt-axis adjustment notch, a movement of the x-y motion stage along the one axis results in a movement of the tilt motion stage along the tilt-axis slanted linear bearing, thereby resulting in a change in the tilt of the tilt motion stage.

5. The motion platform of claim 2,
wherein the motion platform further comprises: a tip-axis adjustment pawl on the base;
wherein the tilt motion stage is a tip and tilt (TnT) motion stage and further comprises:
one or more tip-axis goniometers with different slopes relative to one axis of the x-axis and the y-axis, and
a tip-axis adjustment notch, and
wherein when the tip-axis adjustment pawl is engaged with the tip-axis adjustment notch, a movement of the x-y motion stage along the one axis results in a movement of the TnT motion stage along the one or more tip-axis goniometers, thereby resulting in a change in the tip of the TnT motion stage.

6. A motion platform comprising:
a base;
an x-y motion stage on the base;
a first-axis adjustment engagement component on the base;
a second motion stage on the x-y motion stage and comprising:
a sample carrier,
a first-axis goniometer or bearing, and
a first-axis complementary adjustment engagement component,
wherein when the first-axis adjustment component is engaged with the first-axis complementary adjustment engagement component, a movement of the x-y motion stage along one axis of the x-axis and the y-axis results in a movement of the second motion stage along the first-axis goniometer or bearing, thereby resulting in a change in the first-axis of the second motion stage.

7. The motion platform of claim 6,
wherein the motion platform further comprises a second-axis adjustment engagement component,
wherein the second motion stage further comprises:
a second-axis goniometer or bearing, and
a second-axis complementary adjustment engagement component, and
wherein when the second-axis adjustment engagement component is engaged with the second-axis complementary adjustment engagement component, a movement of the x-y motion stage along the one axis results in a movement of the second motion stage along the second-axis goniometer or bearing, thereby resulting in a change in the second-axis of the second motion stage.

8. The motion platform of any one of claims 6-7, wherein the first-axis is the tip-axis, and/or wherein the second-axis is the tilt-axis.

9. The motion platform of any one of claims 6-7, wherein the first-axis is the tilt-axis, and/or wherein the second-axis is the tip-axis.

10. The motion platform of any one of claims 6-9, wherein the first-axis goniometer or bearing comprises one or more first-axis goniometers.

11. The motion platform of any one of claims 6-9, wherein the first-axis goniometer or bearing comprises a first-axis slanted linear bearing.

12. The motion platform of any one of claims 1-11, wherein the motion platform comprises an x-axis motor and a y-axis motor on the base, and wherein the x-axis motor and y-axis motor move the x-y motion stage along the x-axis and the y-axis of the base, respectively, optionally wherein the motion platform comprises no additional motor other than the x-axis motor and the y-axis motor for changing the tip and/or tilt of the TnT motion stage.

13. The motion platform of any one of claims 1-12, wherein the x-axis motor is a servomotor and/or the y-axis motor is a servomotor.

14. The motion platform of any one of claims 1-13, wherein the tip-axis adjustment pawl and the tilt-axis adjustment pawl point in the opposite directions.

15. The motion platform of any one of claims 1-14, wherein the tip-axis adjustment pawl and the tilt-axis adjustment pawl are elevated from the base.

16. The motion platform of any one of claims 1-15, wherein the tip-axis adjustment pawl and the tilt-axis adjustment pawl are at different heights relative to the base, and wherein the tilt-axis adjustment notch and the tip-axis adjustment notch are at different heights relative to the base.

17. The motion platform of any one of claims 1-15, wherein the tip-axis adjustment pawl and the tilt-axis adjustment pawl are at an identical height relative to the base, and wherein the tilt-axis adjustment notch and the tip-axis adjustment notch are at an identical height relative to the base.

18. The motion platform of any one of claims 1-17, wherein the TnT motion stage comprises no motor.

19. The motion platform of any one of claims 1-18, wherein the TnT motion stage is in contact with the x-y motion stage via the two tip-axis goniometers and the tilt-axis slanted linear bearing.

20. The motion platform of any one of claims 1-18, wherein the TnT motion stage is in contact with the x-y motion stage only via the two tip-axis goniometers and the tilt-axis slanted linear bearing.

21. The motion platform of any one of claims 1-20, wherein the two tip-axis goniometers have different slopes relative to the x-axis.

22. The motion platform of any one of claims 1-20, wherein the slopes of the two tip-axis goniometers have different absolute angles.

23. The motion platform of any one of claims 1-20, wherein the slopes of the two tip-axis goniometers have an identical absolute angle.

24. The motion platform of any one of claims 1-23, wherein the absolute angle of the slope of one or each of the two tip-axis goniometers is about 3.6°.

25. The motion platform of any one of claims 1-24, wherein one or each of the two tip-axis goniometers is at or adjacent to a side surface of the TnT motion platform.

26. The motion platform of any one of claims 1-25, wherein one or each of the two tip-axis goniometers comprises a journal and a slanted pin, optionally wherein the material of the journal comprises bronze, and optionally wherein the material of the pin comprises stainless steel.

27. The motion platform of claim 26, wherein one or each of the two tip-axis goniometers comprises a magnet which retains contact between the journal and the slanted pin.

28. The motion platform of any one of claims 1-27, wherein the tilt-axis slanted linear bearing comprises a linear bearing carriage and a slanted linear bearing rail.

29. The motion platform of any one of claims 1-28, wherein the linear bearing motion angle of the tilt-axis slanted linear bearing is about 3.4°.

30. The motion platform of any one of claims 1-29, wherein the tilt-axis slanted linear bearing is at or adjacent a side surface of the TnT motion platform.

31. The motion platform of any one of claims 1-30, wherein the TnT motion stage comprises a radial bearing in contact with a radial bearing rail, optionally wherein a material of the radial bearing comprises stainless steel, optionally wherein a material of the radial bearing rail comprises stainless steel, optionally wherein the radial bearing rail is co-planar with the x-axis.

32. The motion platform of claim 31, wherein the TnT motion stage comprises at least one magnet which retains contact between the radial bearing and the radial bearing rail, optionally wherein the at least one magnet comprises two magnets.

33. The motion platform of any one of claims 31-32, wherein the radial bearing is at or adjacent to a side surface.

34. The motion platform of any one of claims 1-33, wherein the TnT motion stage comprises 8 side surfaces.

35. The motion platform of any one of claims 1-34, wherein when the tip-axis adjustment pawl is engaged with the tip-axis adjustment notch, the tilt-axis adjustment pawl is not engaged with the tilt-axis adjustment notch, and wherein when the tilt-axis adjustment pawl is engaged with the tilt-axis adjustment notch, the tip-axis adjustment pawl is not engaged with the tip-axis adjustment notch.

36. The motion platform of any one of claims 1-35, wherein when the tip-axis adjustment pawl is engaged with the tip-axis adjustment notch, a movement of the x-y motion stage along the x-axis results in a movement of the TnT motion stage along the two tip-axis goniometers, thereby resulting in a change in the tip of the TnT motion stage

37. The motion platform of any one of claims 1-36, wherein when the tilt-axis adjustment pawl is engaged with the tilt-axis adjustment notch, a movement of the x-y motion stage along the x-axis results in a movement of the TnT motion stage along the tilt-axis slanted linear bearing, thereby resulting in a change in the tilt of the TnT motion stage

38. The motion platform of any one of claims 1-37, wherein when the TnT motion stage is moved along the y-axis to the edge of its travel in one direction of the y-axis, the tip-axis adjustment pawl engages with the tip-axis adjustment notch, and wherein when the TnT motion stage is moved along the y-axis to the edge of its travel in the other direction of the y-axis, the tilt-axis adjustment pawl engages with the tilt-axis adjustment notch.

39. An instrument comprising:
a sensor;
optics; and
a motion platform of any one of claims 1-38.

40. The instrument of claim 39, wherein the instrument comprises a fluorescent imaging system and/or wherein the instrument comprises an optical genome mapping (OGM) system.

41. The instrument of any one of claims 39-40 wherein the base of the motion platform is fixed in position within the motion platform.

42. The instrument of any one of claims 39-41, wherein the motion platform is suspended within the instrument.

43. A method of positioning a sample comprising:
providing a sample;
placing the sample on a sample carrier of a TnT motion stage of a motion platform of any one of claims 1-38;
engaging the tip-axis adjustment paw with the tip-axis adjustment notch;
moving the x-y motion stage along one axis of the x-axis and the y-axis, thereby changing the tip of the TnT motion stage;
engaging the tilt-axis adjustment paw with the tilt-axis adjustment notch; and
moving the x-y motion stage along the one axis of the x-axis and the y-axis, thereby changing the tilt of the TnT motion stage.

44. The method of claim 43, wherein engaging the tip-axis adjustment paw with the tip-axis adjustment notch and moving the x-y motion stage along the one axis of the x-axis and the y-axis occurs before engaging the tilt-axis adjustment paw with the tilt-axis adjustment notch and moving the x-y motion stage along the one axis of the x-axis and the y-axis, thereby changing the tilt of the TnT motion stage.

45. The method of claim 43, wherein engaging the tip-axis adjustment paw with the tip-axis adjustment notch and moving the x-y motion stage along the one axis of the x-axis and the y-axis occurs after engaging the tilt-axis adjustment paw with the tilt-axis adjustment notch and moving the x-y motion stage along the one axis of the x-axis and the y-axis, thereby changing the tilt of the TnT motion stage.

46. The method of any one of claims 43-45, further comprising: changing the x-y position of the motion stage.

47. A method of imaging a sample comprising:
positioning a sample using a method of any one of claims 43-46; and
rastering, using the x-y motion stage, to different positions along the x-axis and/or the y-axis and capturing images of the sample at the different positions.

48. The method of any one of claims 43-47, wherein the sample comprises nucleic acids.

49. The method of claim 48,
wherein the nucleic acids comprise deoxyribonucleic acid (DNA),
wherein the nucleic acids comprise genomic DNA,
wherein the nucleic acids comprise fragmented genomic DNA,
wherein the nucleic acids comprise ribonucleic acids (RNA), and/or
wherein the nucleic acids comprise DNA derived from DNA or RNA.

50. The method of any one of claims 48-49, wherein the sample comprises labeled nucleic acids, optionally wherein the sample comprises fluorescently labeled nucleic acids.
